SEQUENCE LISTING (46 sequences)

SEQ ID NO: 1 - protein, 516 aa, Human alphaherpesvirus 3
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLA

SEQ ID NO: 2 - protein, 516 aa, Human alphaherpesvirus 3
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSYVDHFVNAIEERGFPPTAGQPPATTKPKEITPVNPGTSPLIRYAAWTGGLA

SEQ ID NO: 3 - protein, 516 aa, Human alphaherpesvirus 3
SVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLA

SEQ ID NO: 4 - protein, 516 aa, Human alphaherpesvirus 3
SVLRYDDFHTDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLLRYAAWTGGLA

SEQ ID NO: 5 - protein, 516 aa, Human alphaherpesvirus 3
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPMAGQPPATTKPKEITPVNPGTSPLIRYAAWTGGLA

SEQ ID NO: 6 - protein, 516 aa, Human alphaherpesvirus 3
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLA

SEQ ID NO: 7 - protein, 516 aa, Human alphaherpesvirus 3
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRVPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLA

SEQ ID NO: 8 - protein, 30 aa, Human alphaherpesvirus 3
MGTVNKPVVGVLMGFGIITGTLRITNPVRA

SEQ ID NO: 9 - protein, 8 aa, synthetic construct
GSHHHHHH

SEQ ID NO: 10 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEERGFPPTAGQPPATTKPKEITPVNPGTSPLLR

SEQ ID NO: 11 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEECGFPPTAGQPPATTKPKEITPVNPGTSPLLR

SEQ ID NO: 12 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 13 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLCEISYRFQGKKEADQPWIVVNTSTLFDELELDPPECEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 14 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPCQRIYGVRYTETWSFLPSLTCTGDAAPAIQHICLKHTTCFQDVVVDVDCAENTKEDQLAEISYRFQGKKEADQPWIVVNTSTLFDELELDPPEIEPCVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 15 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRCYGCRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 16 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWCFLPSLTCTGDAAPAIQHICLKHTTCFQDVVVDVDCAENTKEDCLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 17 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYCETWSFLPSLTCTGDAAPAIQHICLKHTTCFQDVVVDVDCAENTKEDQLAEICYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 18 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTECWSFLPSLTCTGDAAPAIQHICLKHTTCFQDVVVDVDCAENTKEDQLACISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 19 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIECSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLCTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 20 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGCRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSCGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 21 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISCRFQGKKEADQPCIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 22 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPCQRIYGVRYTETWSFLPSLTCTGDAAPAIQHICLKHTTCFQDVVVDVDCAENTKEDQLCEISYRFQGKKEADQPWIVVNTSTLFDELELDPPECEPCVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 23 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYCETWSFLPSLTCTGDAAPAIQHICLKHTTCFQDVVVDVDCAENTKEDQLCEICYRFQGKKEADQPWIVVNTSTLFDELELDPPECEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 24 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTECWSFLPSLTCTGDAAPAIQHICLKHTTCFQDVVVDVDCAENTKEDQLCCISYRFQGKKEADQPWIVVNTSTLFDELELDPPECEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 25 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPCQRIYGVRYCETWSFLPSLTCTGDAAPAIQHICLKHTTCFQDVVVDVDCAENTKEDQLAEICYRFQGKKEADQPWIVVNTSTLFDELELDPPEIEPCVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 26 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPCQRIYGVRYTECWSFLPSLTCTGDAAPAIQHICLKHTTCFQDVVVDVDCAENTKEDQLACISYRFQGKKEADQPWIVVNTSTLFDELELDPPEIEPCVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 27 - protein, 507 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYCECWSFLPSLTCTGDAAPAIQHICLKHTTCFQDVVVDVDCAENTKEDQLACICYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLLR

SEQ ID NO: 28 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEERGFPPTAGQPPATTKPKEITPVNPGTSPLIRYAAWTGGLA

SEQ ID NO: 29 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSYVDHFVNAIEECGFPPTAGQPPATTKPKEITPVNPGTSPLIRYAAWTGGLA

SEQ ID NO: 30 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 31 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLCEISYRFQGKKEADQPWIVVNTSTLFDELELDPPECEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 32 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPCQRIYGVRYTETWSFLPSLTCTGDAAPAIQHICLKHTTCFQDVVVDVDCAENTKEDQLAEISYRFQGKKEADQPWIVVNTSTLFDELELDPPEIEPCVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 33 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRCYGCRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 34 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYTETWCFLPSLTCTGDAAPAIQHICLKHTTCFQDVVVDVDCAENTKEDCLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 35 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYCETWSFLPSLTCTGDAAPAIQHICLKHTTCFQDVVVDVDCAENTKEDQLAEICYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 36 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYTECWSFLPSLTCTGDAAPAIQHICLKHTTCFQDVVVDVDCAENTKEDQLACISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 37 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIECSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLCTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 38 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGCRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSCGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSTVDHFVNAIEECGFPPTAGQPPATTKPKEITPVNPGTSPLIRYAAWTGGLA

SEQ ID NO: 39 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISCRFQGKKEADQPCIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 40 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPCQRIYGVRYTETWSFLPSLTCTGDAAPAIQHICLKHTTCFQDVVVDVDCAENTKEDQLCEISYRFQGKKEADQPWIVVNTSTLFDELELDPPECEPCVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 41 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYCETWSFLPSLTCTGDAAPAIQHICLKHTTCFQDVVVDVDCAENTKEDQLCEICYRFQGKKEADQPWIVVNTSTLFDELELDPPECEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 42 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYTECWSFLPSLTCTGDAAPAIQHICLKHTTCFQDVVVDVDCAENTKEDQLCCISYRFQGKKEADQPWIVVNTSTLFDELELDPPECEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 43 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPCQRIYGVRYCETWSFLPSLTCTGDAAPAIQHICLKHTTCFQDVVVDVDCAENTKEDQLAEICYRFQGKKEADQPWIVVNTSTLFDELELDPPEIEPCVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 44 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPCQRIYGVRYTECWSFLPSLTCTGDAAPAIQHICLKHTTCFQDVVVDVDCAENTKEDQLACISYRFQGKKEADQPWIVVNTSTLFDELELDPPEIEPCVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 45 - protein, 516 aa, synthetic construct
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYCECWSFLPSLTCTGDAAPAIQHICLKHTTCFQDVVVDVDCAENTKEDQLACICYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSCCLYHPNAPQCLSHMNSGCTFTSPHLAQRVASTVYQNCEHADNYTAYCLGISHMEPSFGLILCDGGTTLCFVDTPESLSGLYVFVVYFNGHVEAVAYTVVSYVDHFVNAIEECGFPPTAGQPPATTKPKEITPVNPGTSPLIRYAAWTGGLA

SEQ ID NO: 46 - protein, 216 aa, Human alphaherpesvirus 3
AVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLA

(19) United States
(12) Patent Application Publication
(10) Pub. No.: US 2025/0325656 A1
(43) Pub. Date: Oct. 23, 2025

(54) MODIFIED VARICELLA ZOSTER VIRUS GLYCOPROTEIN E PROTEINS

(71) Applicant: GLAXOSMITHKLINE BIOLOGICALS SA, Rixensart (BE)

(72) Inventors: Wayne HARSHBARGER, Rockville, MD (US); Newton Muchugu WAHOME, Rockville, MD (US); Genevieve Anne HOLZAPFEL, Rockville, MD (US)

(73) Assignee: GLAXOSMITHKLINE BIOLOGICALS SA, Rixensart (BE)

(21) Appl. No.: 18/866,641

(22) PCT Filed: May 18, 2023
(86) PCT No.: PCT/IB2023/055123

§ 371 Date: Nov. 18, 2024

Related U.S. Application Data
(60) Provisional application No. 63/344,157, filed on May 20, 2022.

(51) Int. Cl. A61K 39/25; A61P 37/04; C07K 14/005

(52) U.S. Cl. CPC A61K 39/25; A61P 37/04; C07K 14/005; C12N 2760/20222; C12N 2760/20234; C12N 2760/20271

(57) ABSTRACT

Described are modified Varicella Zoster Virus glycoprotein E (VZV gE) proteins having improved stability and/or immunogenicity compared to a non-modified VZV gE. Also described are associated nucleic acids, immunogenic compositions, and methods of using such modified VZV gE proteins in the treatment or prevention of shingles.

### FIELD OF THE INVENTION

The present disclosure relates to modified Varicella Zoster Virus glycoprotein E (VZV gE) proteins having improved characteristics, and their use in an immunogenic compositions or vaccine compositions.

### BACKGROUND TO THE INVENTION

Herpes Zoster (HZ), also known as shingles, is a common and often debilitating disease that occurs primarily in older or immunocompromised individuals. The virus is usually acquired during childhood as chickenpox. Acute symptoms include a painful rash and blisters, fever, light sensitivity, and itching. Long-term complications can include post-herpetic neuralgia (PHN), which manifests as intense nerve pain lasting long after resolution of the acute symptoms. HZ is caused by the symptomatic reactivation of latent varicella zoster virus (VZV) in the dorsal root and cranial ganglia.

Humans vaccinated against varicella zoster virus exhibit protection from HZ and PHN. SHINGRIX, an adjuvanted subunit vaccine containing the VZV glycoprotein E (VZV gE), is marketed for the prevention of HZ and PHN.

Protein instability is an inherent challenge to biologic products, including recombinant subunit vaccines. Improving the stability of recombinant subunit antigens like VZV gE can improve vaccine shelf life and reduce reliance on cold storage. More stable protein antigens may also exhibit improved immunogenicity. Thus, there remains a need for the provision of modified VZV gE proteins for use in immunogenic compositions. Such modified VZV gE proteins exhibit advantageous characteristics, such as increased thermostability, increased shelf-life, and/or improving immune responses (e.g. level and/or duration of response) and/or reducing the amount of material, such as antigen or adjuvant, required to elicit a desired immune response.

### SUMMARY OF THE INVENTION

The present disclosure provides modified VZV gE proteins with improved characteristics compared to non-modified VZV gE proteins, and methods of making and using such proteins. The modified VZV gE proteins of the present disclosure exhibit one or more improved properties selected from: increased stability, such as increased thermostability; increase shelf-life; and improved immune response(s) when administered to a subject. Other benefits may include reducing the amount of material, such as VZV gE antigen or adjuvant, required to elicit a desired immune response.

In one aspect of the present disclosure is provided a modified VZV gE protein comprising at least one cysteine pair capable of forming a non-native disulfide bridge.

In another aspect is provided a modified VZV gE protein comprising at least one cysteine pair capable of forming a non-native disulfide bridge, the cysteine pair selected from the group consisting of: a) Cys-365 and Cys-477, b) Cys-427 and Cys-434, c) Cys-216 and Cys-251, d) Cys-158 and Cys-254, e) Cys-161 and Cys-164, f) Cys-171 and Cys-214, g) Cys-167 and Cys-219, h) Cys-169 and Cys-217, i) Cys-144 and Cys-287, j) Cys-164 and Cys-277, and k) Cys-220 and Cys-232; wherein the cysteines are numbered with respect to SEQ ID NO: 1.

In another aspect the modified VZV gE protein comprises an amino acid sequence at least 85%, 86%, 87%, 88%, 89%, 90%, 91%, 92%, 93%, 94%, 95%, 96%, 97%, 98%, or 99% identical to a non-modified VZV gE protein, such as the non-modified VZV gE protein of any one SEQ ID NOs: 1-7.

In another aspect is provided a modified VZV gE protein comprising an amino acid sequence of any one of any one of SEQ ID NOs: 10-45 or an amino acid sequence at least 85%, 86%, 87%, 88%, 89%, 90%, 91%, 92%, 93%, 94%, 95%, 96%, 97%, 98%, or 99% identical to any one of SEQ ID NOs: 10-45.

In another aspect is provided a nucleic acid, such as a DNA or RNA molecule, encoding a modified VZV gE protein of the present disclosure.

In another aspect is provided an immunogenic composition comprising a modified VZV gE protein or nucleic acid of the present disclosure, and optionally an adjuvant.

In another aspect is provided a method of enhancing an immune response to VZV in a subject comprising administering a modified VZV gE protein, nucleic acid or immunogenic composition of the present disclosure to the subject.

### BRIEF DESCRIPTION OF THE DRAWINGS

FIG. 1: Alignment of ectodomains of 7 non-modified VZV gE sequences. Amino acids differing from the consensus sequence are boxed.

FIG. 2: SDS-PAGE (left) and Western Blot (right) for SEC peak corresponding to VZV gE gIBD-Fab 1A2 complex under reducing and non-reducing conditions.

FIG. 3: Crystal structure of VZV gE gIBD complexed with Fab1E3.

FIG. 4: Crystal structure of VZV gE FcBD (“gE216”) complexed to Fab5A2.

FIG. 5: NanoDSF analysis for non-modified VZV gE (WT1), and modified VZV gE designs based on the structure of the gE Fc Binding Domain (FcBD): Des-1 (disulfide bridge introduced by substitutions H427C and K434C), Des-2 (disulfide bridge introduce by substitutions T365C and R477C, and Des-3 (the combination of the disulfide bridges of Des-1 and Des-2: substitutions H427C, K434C, T365C and R477C). All three designs with non-native disulfide bridges in the FcBD exhibited increased thermostability relative to non-modified VZV gE.

FIG. 6: NanoDSF analysis for non-modified VZV gE (WT2), and modified VZV gE designs based on the structure of the gE FcBD and gIBD. See Example 7 for description of designs Des-4, Des-5, Des-8 and Des-9. All four designs with non-native disulfide bridges in the FcBD and gIBD exhibited increased thermostability relative to non-modified VZV gE.

FIG. 7: NanoDSF analysis for non-modified VZV gE (WT2), and modified VZV gE designs based on the structure of the gE FcBD and gIBD. See Example 8 for description of designs Des-5 (3 non-native disulfide bridges) and designs Des-13, Des-14, Des-15, Des-16, Des-17, and Des-18 (each having 4 non-native disulfide bridges). All designs containing non-native disulfide bridges exhibited increased thermostability relative to non-modified VZV gE. Designs containing 4 non-native disulfide bridges (Des-13, -14, -15, -16, -17 and -18) showed greater thermostability than Des-5 with 3 non-native disulfide bridges.

FIG. 8: Serum anti-gE IgG binding titers at day 35. Data is presented in log 10. To aid visualization, the Y axis is truncated. Individual data points are shown as dots. Error bars denote geometric mean titer (GMT)+/−95% CI. Asterisks denote statistically significant differences compared with wild type at the same dose.

### DETAILED DESCRIPTION

The present disclosure provides the crystal structure of portions of the VZV gE protein, specifically the Fc Binding Domain (FcBD) and the glycoprotein I Binding Domain (gIBD). By analyzing the crystal structure of VZV gE, the inventors have identified an intrinsic flexibility of the non-modified protein. Furthermore the inventors have identified locations within the structure of VZV gE which are amenable to amino acid substitutions which enhance thermostability and immunogenicity by decreasing the conformational flexibility of the VZV gE protein. Thus the present disclosure provides modified VZV gE proteins having improved thermostability and immunogenicity relative to wild-type gE.

### Definitions

Unless otherwise noted, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. The term “at least one” refers to one or more.

Unless specifically stated, as used herein, the term “about” is understood as within a range of normal tolerance in the art. In one embodiment, the term “about” means within 10% of the reported numerical value of the number with which it is being used, such as within 5% of the reported numerical value. For example, the term “about” can be immediately understood as within 10%, 9%, 8%, 7%, 6%, 5%, 4%, 3%, 2%, 1%, 0.5%, 0.1%, 0.05%, or 0.01% of the stated value.

The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Additionally, numerical limitations given with respect to concentrations or levels of a substance, such as solution component concentrations or ratios thereof, and reaction conditions such as temperatures, pressures, and cycle times are intended to be approximate. Unless specified otherwise, where a numerical range is provided, it is inclusive, i.e., the endpoints are included.

For the purposes of the descriptions herein, the abbreviations used for the genetically encoded amino acids are conventional and are as follows:

TABLE 1

| AMINO ACID | THREE-LETTER | ONE-LETTER |
|---|---|---|
| ALANINE | ALA | A |
| ARGININE | ARG | R |
| ASPARAGINE | ASN | N |
| ASPARTATE | ASP | D |
| CYSTEINE | CYS | C |
| GLUTAMATE | GLU | E |
| GLUTAMINE | GLN | Q |
| GLYCINE | GLY | G |
| HISTIDINE | HIS | H |
| ISOLEUCINE | ILE | I |
| LEUCINE | LEU | L |
| LYSINE | LYS | K |
| METHIONINE | MET | M |
| PHENYLALANINE | PHE | F |
| PROLINE | PRO | P |
| SERINE | SER | S |
| THREONINE | THR | T |
| TRYPTOPHAN | TRP | W |
| TYROSINE | TYR | Y |
| VALINE | VAL | V |

“Amino acid” or “residue” as used in the context of the polypeptides disclosed herein refers to the specific monomer at a sequence position (e.g., P5 indicates that the “amino acid” or “residue” at position 5 is a proline.)

“Amino acid difference” or “residue difference” or “amino acid substitution” refers to a change in the residue at a specified position of a polypeptide sequence when compared to a reference sequence (e.g. P5Y indicates that the proline at position 5 of a reference sequence is changed to tyrosine).

“Conservative” amino acid substitutions or mutations refer to the interchangeability of residues having similar side chains, and thus typically involves the substitution of the amino acid in the polypeptide with amino acids within the same or similar defined class of amino acids. However, as used herein, in some embodiments, conservative mutations do not include substitutions from a hydrophilic to hydrophilic, hydrophobic to hydrophobic, hydroxyl-containing to hydroxyl-containing, or small to small residue, if the conservative mutation can instead be a substitution from an aliphatic to an aliphatic, non-polar to non-polar, polar to polar, acidic to acidic, basic to basic, aromatic to aromatic, or constrained to constrained residue. Further, as used herein, A, V, L, or I can be conservatively mutated to either another aliphatic residue or to another non-polar residue. Table 2 below shows exemplary conservative substitutions.

TABLE 2

| Residue | Possible Conservative Mutations |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| P | none |
| N, Q, S, T | Other polar |
| H, Y, W, F | Other aromatic (H, Y, W, F) |
| C | None |

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. Methods of comparing a sequence with a specified reference sequence are known to a skilled person. For example, the Needleman Wunsch method can be used to compare any amino acid or polynucleotide sequence with a reference sequence.

“Corresponding amino acid position” is a term that is widely used and well-understood by a skilled person. A corresponding amino acid position can be identified by aligning the amino acid sequences using any of the well-known amino acid alignment methods. For example, the NCBI BLAST algorithm method can be used to identify a corresponding amino acid position.

“Comprise” (“comprising” or “comprises”) as used herein is open-ended and means “including, but not limited to.” “Having” is used herein as a synonym of comprising. It is understood that wherever embodiments are described herein with the language “comprising,” such embodiments encompass those described in terms of “consisting of” and/or “consisting essentially of.”

“Cysteine pair” as used herein refers to two cysteine residues present in the same VZV gE protein, capable of forming a disulfide bridge between the cysteine members of the pair.

“Deletion” refers to modification of the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, up to 20% of the total number of amino acids, or up to 30% of the total number of amino acids making up the polypeptide. Deletions can be directed to the internal portions and/or terminal portions of the polypeptide. In various embodiments, the deletion can comprise a continuous segment or can be discontinuous.

“Fragment” as used herein, refers to a polypeptide that has an amino-terminal and/or carboxy-terminal deletion, but where the remaining amino acid sequence is identical to the corresponding positions in the sequence. Fragments can be at least 50 amino acids long, at least 100 amino acids long, at least 150 amino acids long or longer, and up to 70%, 80%, 90%, 95%, 98%, and 99%, or more, of the full-length VZV gE protein.

“Improved stability” as used herein means that a modified VZV gE protein is more resistant to denaturation, aggregation, precipitation, and/or adsorption than a non-modified VZV gE reference protein. A widely accepted indicator of protein stability is the melting temperature (Tm) of the protein, which is the temperature at which the protein denatures. Tm can be measured by any method known in the art, including but not limited to differential scanning fluorimetry (DSF), differential static light scattering (DSLS) and isothermal denaturation. See, e.g., Senisterra and Finerty, Mol. BioSyst., 2009, 5, 217-223. In one embodiment, a modified VZV gE protein of the present disclosure exhibits improved stability if the melting temperature of the modified VZV gE protein is at least 5%, at least 10%, at least 15%, at least 20%, at least 25%, at least 30%, at least 40% or at least 50% higher than the melting temperature of non-modified VZV gE protein as measured by differential scanning fluorimetry. Alternatively, a modified VZV gE protein exhibits improved stability if the melting temperature of the modified VZV gE protein is at least 3 degrees C., at least 4 degrees C., at least 5 degrees C., at least 6 degrees C., at least 7 degrees C., at least 8 degrees C., at least 9 degrees C., at least 10 degrees C., at least 15 degrees C., at least 20 degrees C., or at least 25 degrees C. higher than the melting temperature of non-modified VZV gE protein as measured by differential scanning fluorimetry.

“Immunogenicity” is used herein to refer to an antigen's ability to induce an immune response. See generally, e.g., Ma et al., 2011 PLoS Path. 7(9), e1002200. “Improved immunogenicity” as used herein means that a modified VZV gE protein elicits a greater VZV-specific immune response when administered to a subject than a non-modified VZV gE protein. In some embodiments, the modified VZV gE protein elicits a greater T cell response or B cell response compared to a non-modified VZV gE protein. In some embodiments a modified VZV gE protein exhibits improved immunogenicity if it stimulates anti-VZV gE antibody titers in a subject that are at least 5%, at least 10%, at least 15%, at least 20%, at least 25%, at least 30%, at least 40% or at least 50% higher than the anti-VZV gE titers elicited by non-modified VZV gE protein.

“Percentage of sequence identity,” “percent identity,” and “percent identical” are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see, e.g., Altschul, et al., 1990, J. Mol. Biol. 215: 403-410 and Altschul, et al., 1977, Nucleic Acids Res. 3389-3402). Software for performing BLAST analyses is publicly available through the National Center for Biotechnology Information website.

Numerous other algorithms are available that function similarly to BLAST in providing percent identity for two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel, et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison WI), using default parameters provided. The ClustalW program is also suitable for determining identity.

“Protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

“Nucleic acid” herein means a polymeric form of nucleotides of any length, which contain deoxyribonucleotides, ribonucleotides, and/or their analogs. It includes DNA, RNA and DNA/RNA hybrids. It also includes DNA or RNA analogs, such as those containing modified backbones (e.g. peptide nucleic acids (PNAs) or phosphorothioates) or modified bases. Thus, the nucleic acid of the disclosure includes mRNA, DNA, cDNA, recombinant nucleic acids, branched nucleic acids, plasmids, vectors, etc. Where the nucleic acid takes the form of RNA, it may or may not have a 5′ cap. RNA may be a small, medium, or large RNA. The number of nucleotides per strand of a small RNA is from 10-30 (e.g. siRNAs). A medium RNA contains between 30-2000 nucleotides per strand (e.g. non-self-replicating mRNAs). A large RNA contains at least 2,000 nucleotides per strand e.g. at least 2,500, at least 3,000, at least 4,000, at least 5,000, at least 6,000, at least 7,000, at least 8,000, at least 9,000, or at least 10,000 nucleotides per strand. The molecular mass of a single-stranded RNA molecule in g/mol (or Dalton) can be approximated using the formula: molecular mass=(number of RNA nucleotides)×340 g/mol. RNA can include, in addition to any 5′ cap structure, one or more nucleotides having a modified nucleobase. For instance, an RNA can include one or more modified pyrimidine nucleobases, such as pseudouridine and/or 5 methylcytosine residues. In some embodiments, however, the RNA includes no modified nucleobases, and may include no modified nucleotides i.e. all of the nucleotides in the RNA are standard A, C, G and U ribonucleotides (except for any 5′ cap structure, which may include a 7′ methylguanosine). In other embodiments, the RNA may include a 5′ cap comprising a 7′ methylguanosine, and the first 1, 2 or 3 5′ ribonucleotides may be methylated at the 2′ position of the ribose.

Nucleic acids can be in recombinant form, i.e., a form that does not occur in nature. For example, the nucleic acid may comprise one or more heterologous nucleic acid sequences (e.g., a sequence encoding another antigen and/or a control sequence such as a promoter or an internal ribosome entry site). The nucleic acid may be part of a vector i.e., part of a nucleic acid designed for transduction/transfection of one or more cell types. Vectors may be, for example, “expression vectors,” which are designed for expression of a nucleotide sequence in a host cell, or “viral vectors,” which are designed to result in the production of a recombinant virus or virus-like particle.

“Wild-type VZV gE protein” means a glycoprotein E protein from Varicella Zoster Virus having an amino acid sequence found in naturally circulating strains of VZV, such as the VZV gE proteins disclosed in Genbank Accession numbers Q9J3M8, ABF21714, AAT07749, AEW88980, AQT34120, and ANS12941, the contents of which are hereby incorporated by reference in their entireties.

Wild-type VZV gE comprises a transmembrane glycoprotein of 623 amino acids, including an N-terminal signal sequence of about 30 amino acids, followed by an ectodomain which is exposed on the virion surface, a hydrophobic transmembrane domain and a C-terminal intravirion domain.

“VZV gE ectodomain” as used herein refers to the portion of the glycoprotein exposed on the virion surface. Exemplary non-modified VZV gE ectodomains include the polypeptides provided in SEQ ID NOs: 1 to 7, and immunogenic fragments thereof.

“Non-modified VZV gE protein” as used herein refers to a VZV gE protein that does not contain a non-native disulfide bridge. Non-modified VZV gE proteins may contain one or more native disulfide bridges at cysteine pairs Cys-357/Cys-383, Cys-366/Cys-375, and Cys-402/Cys-412, as numbered according to the wild-type gE ectodomain of SEQ ID NO: 1. Non-modified VZV gE proteins include wild-type VZV gE proteins. Examples of non-modified VZV gE proteins include the sequences described in GenBank Accession Nos. Q9J3M8, ABF21714, AAT07749, AEW88980, AQT34120, and ANS12941; sequence number 6 of International Patent Publication WO2020245207, and the sequences provided herein as SEQ ID NOs: 1-7.

“Modified VZV gE protein” as used herein refers to a VZV gE protein having at least one non-native disulfide bridge relative to non-modified VZV gE protein. A non-native disulfide bridge is one other than those which form in wild-type VZV gE protein between cysteine pairs Cys-357/Cys-383, Cys-366/Cys-375, and Cys-402/Cys-412, as numbered according to SEQ ID NO: 1. Non-native disulfide bridges can be introduced by substituting one or more native amino acid residues with non-native cysteines capable of forming a non-native disulfide bridge. A non-native disulfide bridge can be formed between a native free cysteine and a non-native cysteine; or between two non-native cysteines.

“Native free cysteine” residue as used herein refers to a cysteine residue present in a wild-type VZV gE protein sequence, such as the sequence of SEQ ID NO: 1, which does not form a native disulfide bridge. Examples of native free cysteines include cysteines at positions 178, 190, 196, and 206 of SEQ ID NOs: 1-7.

### Vzv gE Proteins

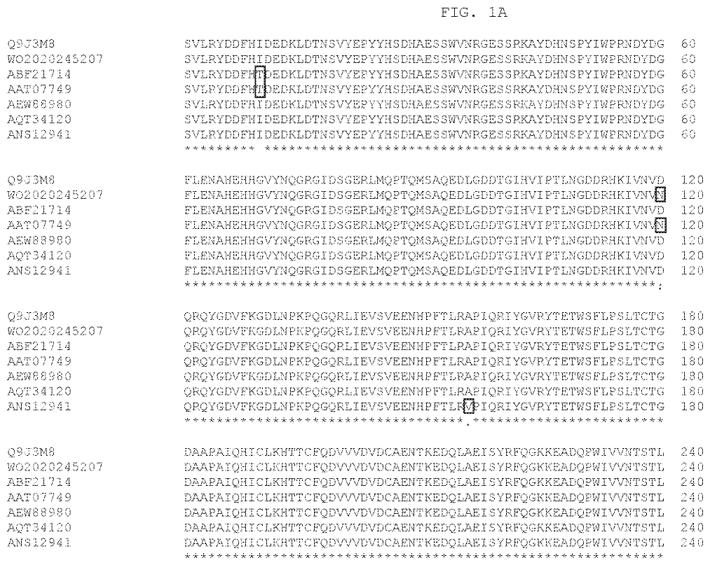

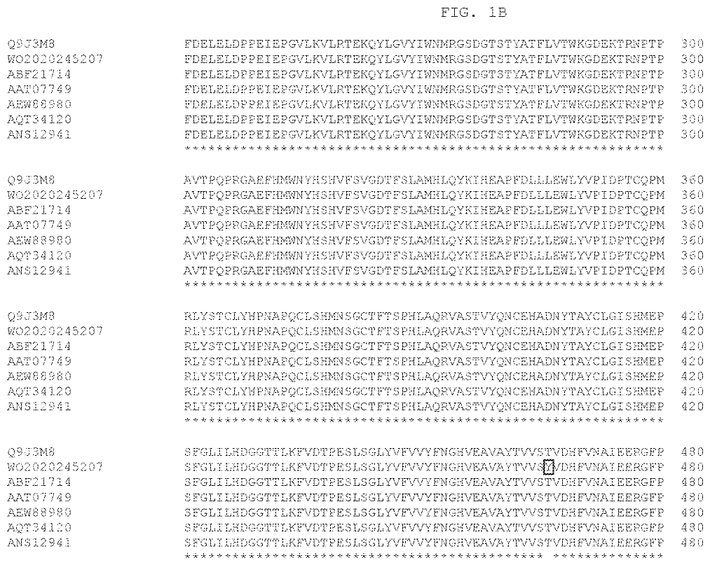

The modified Varicella Zoster glycoprotein E (VZV gE) proteins of the present disclosure contain one or more amino acid substitutions relative to a non-modified VZV gE which confer advantageous properties to the modified protein. Wild-type full-length gE protein comprises a signal peptide, an ectodomain, a hydrophobic anchor region and a C-terminal tail. An adjuvanted recombinant VZV gE subunit vaccine comprising a non-modified VZV gE ectodomain (lacking the hydrophobic anchor and C-terminal tail) is marketed under the brand name SHINGRIX for the prevention of shingles (herpes zoster). (Syed, Recombinant Zoster Vaccine (Shingrix®): A Review in Herpes Zoster, Drugs & Aging (2018) 35:1031-1040). Several non-modified VZV gE variants are known in the art. A non-limiting list of exemplary non-modified VZV gE ectodomain sequences is presented in Table 3. An alignment of the non-modified amino acid sequences is shown in FIG. 1.

TABLE 3
Non-modified VZV gE Proteins

| SEQ | |
|---|---|
| ID No | Accession No. |
| 1 | Genbank Accession No. Q9J3M8 |
| 2 | Patent Pub. No. WO2020245207 (sequence no. 6) |
| 3 | Genbank Accession No. ABF21714 |
| 4 | Genbank Accession No. AAT07749 |
| 5 | Genbank Accession No. AEW88980 |
| 6 | Genbank Accession No. AQT34120 |
| 7 | Genbank Accession No. ANS12941 |

The ectodomain of wild-type VZV gE contains three cysteine pairs which form native disulfide bridges in the wild-type protein. These native disulfide bridges form between Cys-357/Cys-383, Cys-366/Cys-375, and Cys-402/Cys-412, as numbered according to the wild-type gE ectodomain of any one of SEQ ID NOs: 1-7. The ectodomain of wild-type VZV gE also contains cysteine residues which do not form native disulfide bridges in wild-type VZV gE. Such cysteines are referred to as free, or unpaired, cysteines, and are found at positions 178, 190, 196 and 206 of the wild-type VZV gE ectodomain, according to the numbering of any one of SEQ ID Nos: 1-7.

In one embodiment of the present disclosure is provided a modified VZV gE protein comprising an amino acid substitution relative to wild-type VZV gE that introduces a cysteine capable of forming a non-native disulfide bridge with another cysteine in the VZV gE protein. A non-native disulfide bridge can be introduced into the VZV gE protein by substituting a non-cysteine residue with a cysteine residue capable for forming a disulfide bridge with a free (i.e., unpaired) cysteine present in the wild-type sequence. Alternatively, a cysteine pair capable of forming a non-native disulfide bridge can be introduced into the VZV gE protein by substituting two non-cysteine residues with cysteine residues capable of forming a disulfide bridge between them. Such modifications may be referred to herein as “disulfide bridge mutations” with the resulting amino acid being referred to as a “disulfide bridge mutation.” Without wishing to be bound by theory, such mutations are believe to stabilize the protein because the newly introduced disulfide bridge locks (restrains) the VZV gE protein and thereby reduce its dynamics.

In one embodiment is provided a modified VZV gE protein comprising at least one cysteine pair capable of forming a non-native disulfide bridge. In one aspect, one of the cysteines in the cysteine pair is a substitution relative to the corresponding position in wild-type VZV gE protein. In another aspect, both of the cysteines in the cysteine pair are substitutions relative to the corresponding positions in wild-type VZV gE protein.

In another embodiment is provided a modified VZV gE protein comprising at least two cysteine pairs, such as 2, 3, 4, 5, 6, 7, 8, 9, 10 or more cysteine pairs, each cysteine pair capable of forming a non-native disulfide bridge.

In certain embodiments the cysteine pair is in the VZV gE ectodomain. Exemplary VZV gE ectodomains suitable for modification according to the present disclosure are presented in FIG. 1 (SEQ ID Nos: 1-7). In some embodiments a VZV gE ectodomain is truncated by 1, 2, 3, 4, 5, 6, 7, 8, or 9 amino acids at the N- and/or C-terminus. Exemplary VZV gE ectodomains suitable for modification according to the present disclosure include polypeptides comprising amino acids 1 to 516, 1 to 515, 1 to 514, 1 to 513, 1 to 512, 1 to 511, 1 to 510, 1 to 509, 1 to 508, or 1 to 507 of any one of SEQ ID NOs. 1 to 7, or a polypeptide at least 85%, 86%, 87%, 88%, 89%, 90%, 91%, 92%, 93%, 94%, 95%, 96%, 97%, 98%, or 99% identical thereto. Additional VZV gE ectodomains suitable for modification according to the present disclosure include polypeptides comprising amino acids 2 to 516, 3 to 516, 4 to 516, 5 to 516, 6 to 516, 7 to 516, 8 to 516, 9 to 516, or 10 to 516 of any one of SEQ ID NOs. 1 to 7, or a polypeptide at least 85%, 86%, 87%, 88%, 89%, 90%, 91%, 92%, 93%, 94%, 95%, 96%, 97%, 98%, or 99% identical thereto.

In a specific embodiment, a modified VZV gE protein of the present disclosure comprises amino acid residues 1 to 508, or alternatively amino acids 1 to 516, of any one of SEQ ID NOs: 1 to 7, wherein at least one non-cysteine residue is substituted with a cysteine residue to introduce a cysteine pair capable of forming a non-native disulfide bridge.

In one aspect, at least one member of the cysteine pair is in the VZV gE gI Binding Domain (gIBD), represented by amino acid residues 116 to 305 of any one of SEQ ID Nos: 1-7, or a corresponding portion of a wild-type VZV gE protein. In another aspect, at least one member of the cysteine pair is in the VZV gE Fc Binding Domain (FcBD), represented by amino acid residues 306 to 516 of any one of SEQ ID Nos: 1-7, or a corresponding portion of a wild-type VZV gE protein. In additional embodiments, both members of the cysteine pair are located in the gIBD or the FcBD.

In another embodiment is provided a modified VZV gE protein comprising at least one cysteine pair capable of forming a non-native disulfide bridge, the at least one cysteine pair selected from the group consisting of: a) Cys-365 and Cys-477; b) Cys-427 and Cys-434; c) Cys-216 and Cys-251; d) Cys-158 and Cys-254; e) Cys-161 and Cys-164; f) Cys-171 and Cys-214; g) Cys-167 and Cys-219; h) Cys-169 and Cys-217; i) Cys-144 and Cys-287; j) Cys-164 and Cys-277; and k) Cys-220 and Cys-232; wherein the cysteines are numbered with respect to SEQ ID NO: 1.

In one aspect the modified VZV gE protein comprises at least 2, at least 3, at least 4, at least 5, at least 6, at least 7, at least 8, at least 9, at least 10, or all 11 cysteine pairs selected from the group consisting of (a) to (k) above. In another aspect the modified VZV gE protein comprises at least one cysteine pair selected from (a) and (b), and at least one cysteine pair selected from (c) to (k) above.

In another embodiment the modified VZV gE protein comprises the cysteine pairs of a) and b) above; and at least one cysteine pair selected from c) to k) above.

In another embodiment the modified VZV gE protein comprises the cysteine pair of a), above.

In another embodiment the modified VZV gE protein comprises the cysteine pair of b), above.

In another embodiment, the modified VZV gE protein comprises the cysteine pairs of (a) and (b), above.

In another embodiment, the modified VZV gE protein comprises the cysteine pairs of (a), (b) and (c), above.

In another embodiment, the modified VZV gE protein comprises the cysteine pairs of (a), (b) and (d), above.

In another embodiment, the modified VZV gE protein comprises the cysteine pairs of (a), (b) and (e), above.

In another embodiment, the modified VZV gE protein comprises the cysteine pairs of (a), (b) and (f), above.

In another embodiment, the modified VZV gE protein comprises the cysteine pairs of (a), (b) and (g), above.

In another embodiment, the modified VZV gE protein comprises the cysteine pairs of (a), (b) and (h), above.

In another embodiment, the modified VZV gE protein comprises the cysteine pairs of (a), (b) and (i), above.

In another embodiment, the modified VZV gE protein comprises the cysteine pairs of (a), (b) and (j), above.

In another embodiment, the modified VZV gE protein comprises the cysteine pairs of (a), (b) and (k), above.

In another embodiment, the modified VZV gE protein comprises the cysteine pairs of (a), (b), (c) and (d), above.

In another embodiment, the modified VZV gE protein comprises the cysteine pairs of (a), (b), (c) and (f), above.

In another embodiment, the modified VZV gE protein comprises the cysteine pairs of (a), (b), (c) and (g), above.

In another embodiment, the modified VZV gE protein comprises the cysteine pairs of (a), (b), (d) and (f), above.

In another embodiment, the modified VZV gE protein comprises the cysteine pairs of (a), (b), (c) and (g), above.

In another embodiment, the modified VZV gE protein comprises the cysteine pairs of (a), (b), (f) and (g).

In additional embodiments the modified VZV gE protein comprises at least one cysteine pair capable of forming a non-native disulfide bridge, the at least one cysteine pair selected from the group consisting of: Cys-142 and Cys-285; Cys-144 and Cys-287; Cys-146 and Cys-152; Cys-146 and Cys-261; Cys-147 and Cys-150; Cys-154 and Cys-256; Cys-156 and Cys-254; Cys-156 and Cys-158; Cys-156 and cys-271; Cys-158 and Cys-254; Cys-161 and Cys-164; Cys-164 and Cys-277; Cys-164 and Cys-275; Cys-165 and Cys-276; Cys-166 and Cys-275; Cys-167 and Cys-219; Cys-167 and Cys-274; Cys-168 and Cys-273; Cys-169 and Cys-217; Cys-169 and Cys-272; Cys-170 and Cys-215; Cys-171 and Cys-214; Cys-171 and Cys-270; Cys-173 and Cys-268; Cys-216 and Cys-251; Cys-217 and Cys-236; Cys-217 and Cys-235; Cys-220 and Cys-232; Cys-238 and Cys-240; Cys-239 and Cys-242; Cys-241 and Cys-248; Cys-241 and Cys-246; Cys-244 and Cys-246; Cys-250 and Cys-257; Cys-269 and Cys-285; Cys-271 and Cys-283; Cys-275 and Cys-277; Cys-275 and Cys-279; and Cys-289 and Cys-292, wherein the cysteines are numbered with respect to SEQ ID NO: 1.

In another embodiment, a modified VZV gE protein as described herein further comprises a cavity-filling mutation in addition to at least one cysteine pair capable of forming a non-native disulfide bridge. In one aspect the cavity-filling mutation is selected from one or more of the mutations shown in Table 4 (numbered with respect to SEQ ID NO: 1):

TABLE 4
Cavity Filling Mutations

| Wt | | Construct Naming |
|---|---|---|
| Residue | Mutation | Convention |
| 443 | Serine to Tyrosine | gE_S443Y |
| 443 | Serine to Methionine | gE_S443M |
| 443 | Serine to Phenylalanine | gE_S443F |
| 443 | Serine to Isoleucine | gE_S443I |
| 442 | Leucine to Tyrosine | gE_L442Y |
| 442 | Leucine to Phenylalanine | gE_L442F |
| 442 | Leucine to Isoleucine | gE_L442I |
| 342 | Phenylalanine to Histidine | gE_F342H |
| 342 | Phenylalanine to Methionine | gE_F342M |
| 342 | Phenylalanine to Threonine | gE_F342T |
| 342 | Phenylalanine to Alanine | gE_F342A |
| 429 | Glycine to Tryptophan | gE_G429W |
| 429 | Glycine to Methionine | gE_G429M |
| 409 | Threonine to Methionine | gE_T409M |
| 409 | Threonine to Valine | gE_T409V |
| 409 | Threonine to Isoleucine | gE_T409I |
| 409 | Threonine to Leucine | gE_T409L |
| 409 | Threonine to Phenylalanine | gE_T409F |
| 395 | Alanine to Leucine | gE_A395L |
| 395 | Alanine to Valine | gE_A395V |
| 395 | Alanine to Methionine | gE_A395M |
| 395 | Alanine to Phenylalanine | gE_A395F |
| 337 | Isoleucine to Methionine | gE_I337M |
| 337 | Isoleucine to Phenylalanine | gE_I337F |
| 337 | Isoleucine to Leucine | gE_I337L |

In another embodiment, a modified VZV gE protein as described herein comprises combinations of point mutations as shown in Table 5 (numbered with respect to SEQ ID NO: 1):

TABLE 5
Consensus Sequence Designs

| | | Construct |
|---|---|---|
| | | Naming |
| Wt Residue(s) | Mutation(s) | Convention |
| 356, 390, 408 | T356R, L390W, Y408W | gE_neg3 |
| 356, 379, 381, 382, | T356S, M379E, S381A, G382K, | gE_neg1_5 |
| 390, 392, 400, 408, | L390W, Q392L, Q400P, Y408W, | |
| 409, 434, 437, 479 | T409W, K434V, D437N, F479L | |
| 331, 339, 340, 356, | M331V, E339D, A340E, T356R, | gE_neg075 |
| 358, 379, 380, 381, | Q358G, M379E, N380D, S381A, | |
| 382, 390, 392, 400, | G382R, L390W, Q392L, Q400P, | |
| 403, 408, 409, 434, | E403T, Y408W, T409W, K434V, | |
| 437, 462, 467, 479 | D437N, T462I, V467L, F479L | |
| 331, 390, 462, 479 | M331V, L390W, T462I, F479L | gE_WH |

In one embodiment is provided a modified VZV gE protein comprising at least one cysteine pair capable of forming a non-native disulfide bridge as described herein, the modified VZV gE protein having an amino acid sequence at least 85%, 86%, 87%, 88%, 89%, 90%, 91%, 92%, 93%, 94%, 95%, 96%, 97%, 98%, or 99% identical to a wild-type VZV gE protein. In one aspect the wild-type VZV gE protein is selected from the group consisting of SEQ ID NOs: 1-7. In another aspect, the wild-type VZV gE protein is SEQ ID NO: 1 or SEQ ID NO: 2.

In another embodiment, the modified VZV gE protein as described herein comprises an amino acid sequence at least 85%, 86%, 87%, 88%, 89%, 90%, 91%, 92%, 93%, 94%, 95%, 96%, 97%, 98%, or 99% identical to an amino acid sequence selected from the group consisting of SEQ ID NOs: 10-27.

In another embodiment, the modified VZV gE protein as described herein comprises an amino acid sequence at least 85%, 86%, 87%, 88%, 89%, 90%, 91%, 92%, 93%, 94%, 95%, 96%, 97%, 98%, or 99% identical to an amino acid sequence selected from the group consisting of SEQ ID NOs: 28-45.

In another embodiment, a modified VZV gE protein as described herein further comprising a signal sequence to promote expression of the protein from a host cell. In one aspect the signal sequence comprises an amino acid sequence at least 90% identical, such as at least 91%, 92%, 93%, 94%, 95%, 96%, 97%, 98%, or 99% identical, to SEQ ID NO: 8.

The modified VZV gE proteins and compositions of the present disclosure exhibit advantageous properties compared to non-modified VZV gE proteins.

In one embodiment, the modified VZV gE proteins and compositions as described herein have improved stability over non-modified VZV gE proteins. In one aspect the modified VZV gE protein has a higher melting temperature (Tm) relative to wild-type VZV gE protein. In certain embodiments, the melting temperature of a modified VZV gE protein of the present disclosure is at least 5%, at least 10%, at least 15%, at least 20%, at least 25%, at least 30%, at least 40% or at least 50% higher than the melting temperature of non-modified VZV gE protein as measured by differential scanning fluorimetry. Alternatively, a modified VZV gE protein exhibits improved stability if the melting temperature of the modified VZV gE protein is at least 3 degrees C., at least 4 degrees C., at least 5 degrees C., at least 6 degrees C., at least 7 degrees C., at least 8 degrees C., at least 9 degrees C., at least 10 degrees C., at least 15 degrees C., at least 20 degrees C., or at least 25 degrees C. higher than the melting temperature of non-modified VZV gE protein as measured by differential scanning fluorimetry.

In another embodiment, the modified VZV gE proteins and compositions described herein elicit an immune response against VZV, such as an immune response against the VZV glycoprotein E (VZV gE). The elicited immune response may be an antigen specific B cell response which produces neutralizing antibodies. The elicited immune response may be an antigen specific T cell response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2.

An immune response may be characterised by various parameters. In one embodiment, the immune response is characterised by the level of response, suitably as indicated by the serum titer of IgG antibodies discussed above level which bind and/or are specific to VZV gE. In certain aspects, the antibodies are serum neutralizing antibodies against VZV gE. In one aspect the level of the immune response elicited by modified VZV gE proteins and compositions is at least equivalent to the immune response elicited by an equivalent amount of non-modified VZV gE protein when administered to a subject. In certain aspects a modified VZV gE protein elicits a greater VZV-specific immune response when administered to a subject than a non-modified VZV gE protein. In some embodiments, the modified VZV gE protein elicits a greater T cell response or B cell response compared to a non-modified VZV gE protein. In some embodiments a modified VZV gE protein stimulates anti-VZV gE antibody titers in a subject that are at least 5%, at least 10%, at least 15%, at least 20%, at least 25%, at least 30%, at least 40% or at least 50% higher than the anti-VZV gE titers elicited by non-modified VZV gE protein.

In a further embodiment, the immune response is characterised by the duration of immune response, for example as indicated by the period of time over which the subject maintains a serum titer of antibodies as discussed above binding and/or specific to VZV gE. In one aspect the duration of the immune response elicited by modified VZV gE proteins and compositions is at least equivalent to the duration of the immune response elicited by an equivalent amount of non-modified VZV gE protein when administered to a subject. In certain aspects the duration of the immune response elicited by modified VZV gE proteins and compositions is increased by at least 1 year, at least 2 years, or at least 3 years compared to an equivalent amount of non-modified VZV gE.

### Nucleic Acids

Also provided are recombinant nucleic acid molecules encoding modified VZV gE proteins described herein. The recombinant nucleic acid molecules of the present invention may be within a vector (an expression vector, for example) and may be operably linked to one or more control element (a promoter and/or an enhancer, for example).

In one embodiment is provided a nucleic acid encoding a modified VZV gE protein as described herein. In one aspect the nucleic acid is deoxyribonucleic acid (DNA). In another aspect the nucleic acid is ribonucleic acid (RNA). RNA encoding modified VZV gE proteins of the present disclosure can be non-replicating RNA or self-replicating RNA. Optionally the self-replicating RNA is an alphavirus replicon. An alphavirus replication particle (VRP) may comprise the alphavirus replicon.

In another embodiment is provided a nucleic acid encoding a modified VZV gE protein comprising at least one cysteine pair capable of forming a non-native disulfide bridge, the cysteine pair selected from the group consisting of:
a) Cys-365 and Cys-477; b) Cys-427 and Cys-434; c) Cys-216 and Cys-251; d) Cys-158 and Cys-254; e) Cys-161 and Cys-164; f) Cys-171 and Cys-214; g) Cys-167 and Cys-219; h) Cys-169 and Cys-217; i) Cys-144 and Cys-287; j) Cys-164 and Cys-277; and k) Cys-220 and Cys-232; wherein the cysteines are numbered with respect to SEQ ID NO: 1.

In one embodiment is provided a nucleic acid encoding a modified VZV gE protein comprising an amino acid sequence at least 85%, 86%, 87%, 88%, 89%, 90%, 91%, 92%, 93%, 94%, 95%, 96%, 97%, 98%, or 99% identical to an amino acid sequence selected from the group consisting of SEQ ID NOs: 10-27.

In another embodiment is provided a nucleic acid encoding a modified VZV gE protein as described herein comprising an amino acid sequence at least 85%, 86%, 87%, 88%, 89%, 90%, 91%, 92%, 93%, 94%, 95%, 96%, 97%, 98%, or 99% identical to an amino acid sequence selected from the group consisting of SEQ ID NOs: 28-45.

The recombinant nucleic acid can be monocistronic or polycistronic. Any suitable DNA or RNA can be used as the nucleic acid vector that carries the open reading frame that encodes the modified VZV gE proteins of the disclosure. Suitable nucleic acid vectors are known in the art and include, for example, plasmids, DNA obtained from DNA viruses such as vaccinia virus vectors (e.g., NYVAC, see U.S. Pat. No. 5,494,807), adenoviral vectors and poxvirus vectors (e.g., ALVAC canarypox vector, Sanofi Pasteur), and RNA obtained from suitable RNA viruses such as alphavirus. If desired, the recombinant nucleic acid molecule can be modified, e.g., contain modified nucleobases and or linkages as described further herein.

Recombinant RNA encoding a modified VZV gE protein can be delivered as naked RNA (e.g. merely as an aqueous solution of RNA) but, to enhance entry into cells and also subsequent intercellular effects, the RNA can also administered in combination with a delivery system, such as a particulate or emulsion delivery system. A large number of delivery systems are well known to those of skill in the art, including for example (i) liposomes, (ii) non-toxic and biodegradable polymer microparticles, and (iii) cationic submicron oil-in-water emulsions.

Also provided herein is a vector comprising a nucleic acid that encodes a modified VZV gE protein as described herein. In one aspect the vector may be an expression vector comprising promoters and terminators suitable for expression within a host cell. Such promoters and terminators have been described in, for example, U.S. Pre-grant Pub. Nos. 2015/0322115 and 2015/0359879.

### Host Cells

The present disclosure also provides a host cell that expresses a nucleic acid molecule as described herein, wherein said cell does not comprise the full VZV genome. The host cell may be stably transformed with said nucleic acid molecule or plurality of nucleic acid molecules of the invention. In certain embodiments, the host cell is a mammalian cell. Suitable mammalian cells include, for example, Chinese hamster ovary (CHO) cells, human embryonic kidney cells (HEK-293 cells, NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, and the like.

### Adjuvants

Vaccine and immunogenic compositions of the present disclosure may comprise an adjuvant in addition to a modified VZV gE protein. The term “adjuvant” refers to a compound that when administered in conjunction with or as part of an immunogenic composition of vaccine of the invention augments, enhances and/or boosts the immune response to a modified VZV gE protein, but when the compound is administered alone does not generate an immune response to the modified VZV gE protein. Adjuvants can enhance an immune response by several mechanisms including, e.g. lymphocyte recruitment, stimulation of B and/or T cells, and stimulation of macrophages. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see United Kingdom Patent GB2220211), MF59 (Novartis), AS03 (GlaxoSmithKline), AS04 (GlaxoSmithKline), and saponins, such as QS21 (see Kensil et al. in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al. N. Engl. J. Med. 336, 86-91 (1997)).

In one embodiment an adjuvant suitable for use in the present disclosure comprises a saponin and a TLR4 agonist. In one aspect the saponin is QS21. In one aspect the TLR4 agonist is 3-O-desacyl-4′-Monophosphoryl Lipid A (3D-MPL). In one embodiment, the saponin and TLR-4 agonist are formulated with liposomes. Liposomes may contain a neutral lipid, for example phosphatidylcholine, which is suitably non-crystalline at room temperature, for example egg yolk phosphatidylcholine, dioleoyl phosphatidylcholine (DOPC) or dilauryl phosphatidylcholine. In a one embodiment, the liposomes of the present invention contain DOPC. The liposomes may also contain a charged lipid which increases the stability of the lipsome-QS21 structure for liposomes composed of saturated lipids.

### Immunogenic Compositions

The present disclosure provides immunogenic compositions and vaccines comprising a modified VZV gE protein as described herein, or a nucleic acid or vector encoding a modified VZV gE protein as described herein.

In one embodiment is provided an immunogenic composition or vaccine comprising a modified VZV gE protein as described herein, and optionally a pharmaceutically acceptable excipient and/or carrier. In one aspect the immunogenic composition or vaccine further comprises an adjuvant.

Immunogenic compositions comprise an immunologically effective amount of the modified VZV gE protein of the present disclosure, as well as any other components. By “immunologically effective amount”, it is meant that the administration of that amount to an individual, either as a single dose regimen or as part of a multi-dose regimen is effective for treatment or prevention. This amount varies depending on the health and physical condition of the individual to be treated, age, the degree of protection desired, the formulation of the vaccine and other relevant factors.

Pharmaceutically acceptable excipients and carriers are described, for example, in Remington's Pharmaceutical Sciences, by E. W. Martin, Mack Publishing Co. Easton, PA, 5th Edition (975). Pharmaceutically acceptable excipients can include a buffer, such as a phosphate buffer (e.g. sodium phosphate). Pharmaceutically acceptable excipients can include a salt, for example sodium chloride. Pharmaceutically acceptable excipients can include a solubilizing/stabilizing agent, for example, polysorbate (e.g. TWEEN 80). Pharmaceutically acceptable excipients can include a preservative, for example 2-phenoxyethanol or thiomersal. Pharmaceutically acceptable excipients can include a carrier such as water or saline.

Also provided is a vaccine comprising an immunogenic composition of the invention and optionally an adjuvant. In one embodiment, the vaccine or immunogenic composition of the invention comprises a modified VZV gE protein antigen and an adjuvant wherein the adjuvant is AS01, an oil-in-water emulsion (e.g., MF59, and AS03 and their respective subtypes including subtypes B and E), an aluminum salt (e.g., aluminum phosphate and aluminum hydroxide), a saponin (e.g. QS21), an agonist of Toll-like receptors (TLRa) (e.g., TLR4a and TLR7a), or a combination thereof (e.g., Alum-TLR7a (Buonsanti et al., Novel adjuvant Alum-Tlr7a significantly potentiates immune response to glycoconjugate vaccines, 2016 Sci. Rep. 6:29063 (DOI: 10.1038/srep29063)). By “TLR agonist” it is meant a component which is capable of causing a signalling response through a TLR Signaling pathway, either as a direct ligand or indirectly through generation of endogenous or exogenous ligand (Sabroe et al, Toll-like Receptors in Health and Disease: Complex Questions Remain, 2003 J. Immunol. 171(4): 1630-1635). A TLR4 agonist, for example, is capable of causing a signalling response through a TLR-4 signalling pathway. A suitable example of a TLR-4 agonist is a lipopolysaccharide, suitably a non-toxic derivative of lipid A, particularly monophosphoryl lipid A or more particularly 3-Deacylated monophosphoryl lipid A (3D-MPL). The adjuvants described herein may be combined with any of the antigen(s) herein described.

In one embodiment, the immunogenic composition or vaccine of the present disclosure is lyophilized.

Also provided is a kit comprising a modified VZV gE protein or immunogenic composition described herein, and optionally an adjuvant. In one aspect the modified VZV gE protein or immunogenic composition is lyophilized, and the adjuvant is liquid.

### Methods of Use

Provided herein are methods of using compositions od the present disclosure, including modified VZV gE proteins, nucleic acids, immunogenic compositions, and kits, for prevention and/or amelioration of herpes zoster in humans and other mammals.

In one embodiment is provided a method of enhancing an immune response to VZV in a subject comprising administering a modified VZV gE protein, a nucleic acid, a vector, or an immunogenic composition of the present disclosure to the subject.

Also provided is a method of preventing or ameliorating herpes zoster (HZ) and/or post herpetic neuralgia (PHN) in a subject comprising administering a modified VZV gE protein, a nucleic acid, a vector, or an immunogenic composition of the present disclosure to the subject. In one aspect, the method comprises administering an immunogenic composition comprising a modified VZV gE protein to the subject. In another aspect the method comprises administering an immunogenic composition comprising a nucleic acid, such as an RNA molecule or a vector, encoding a modified VZV gE protein to the subject.

Vaccines can be administered once, twice, three times, four times or more. In one embodiment, a modified VZV gE protein, a nucleic acid, a vector, or an immunogenic composition of the present disclosure is administered to a subject in a single dose regimen. In another embodiment, a modified VZV gE protein, a nucleic acid, a vector, or an immunogenic composition of the present disclosure is administered to a subject in a two-dose regimen.

In one embodiment, the modified VZV gE protein, nucleic acid, vector, or immunogenic composition of the present disclosure is administered at a lower dose than the effective dose of a composition comprising a non-modified VZV gE protein. In one aspect, an immunogenic composition comprising a modified VZV gE protein of the present disclosure is administered to an adult subject at a dose of less than 50 mcg, for example less than 45 mcg, 40 mcg, 35 mcg, 30 mcg, or 25 mcg of modified VZV gE protein. In another aspect an immunogenic composition comprising a modified VZV gE protein of the present disclosure is administered to an adult subject at a dose of 50%, 60%, 70%, 80% or 90% of the effective dose of a composition comprising a non-modified VZV gE protein.

In another embodiment, the modified VZV gE composition of the present disclosure is administered with a lower dose of adjuvant than a composition comprising an non-modified VZV gE protein. In one aspect, an immunogenic composition of the present disclosure is administered to an adult subject at an adjuvant dose of less than 50 mcg of 3-O-desacyl-4′-Monophosphoryl Lipid A (3D-MPL). In another aspect, the immunogenic composition of the present disclosure is administered to an adult subject at an adjuvant dose of less than 50 mcg of QS21.

### Embodiments

The invention is further disclosed in the following clauses:

Clause 1: A modified Varicella Zoster Virus glycoprotein E (VZV gE) protein comprising at least one cysteine pair capable of forming a non-native disulfide bridge.

Clause 2: The modified VZV gE protein of clause 1, wherein at least one of the cysteines in the at least one cysteine pair is a substitution relative to the corresponding position in a non-modified VZV gE protein.

Clause 3: The modified VZV gE protein of clause 1, wherein both of the cysteines in the at least one cysteine pair are substitutions relative to the corresponding positions in a non-modified VZV gE protein.

Clause 4: The modified VZV gE protein of any one of Clauses 1-2 comprising at least two, at least three, at least four, or at least five, at least six, at least seven at least eight, at least nine or at least ten cysteine pairs, each cysteine pair capable of forming a non-native disulfide bridge.

Clause 5: The modified VZV gE protein of any one of clauses 1-4, wherein the cysteine pair is in the VZV gE ectodomain.

Clause 6: The modified VZV gE protein of any one of clauses 1-5, wherein the cysteine pair is in the VZV gE Fc Binding Domain (FcBD).

Clause 7: The modified VZV gE protein of any one of clauses 1-6, wherein the cysteine pair is in the VZV gE gI Binding Domain (gIBD).

Clause 8: The modified VZV gE protein of clause 1, the at least one cysteine pair selected from the group consisting of: a) Cys-365 and Cys-477, b) Cys-427 and Cys-434, c) Cys-216 and Cys-251, d) Cys-158 and Cys-254, e) Cys-161 and Cys-164, f) Cys-171 and Cys-214, g) Cys-167 and Cys-219, h) Cys-169 and Cys-217, i) Cys-144 and Cys-287, j) Cys-164 and Cys-277, and k) Cys-220 and Cys-232; wherein the cysteines are numbered with respect to SEQ ID NO: 1.

Clause 9: The modified VZV gE protein of clause 8 comprising at least two, at least three, at least four, at least five, at least six, at least seven, at least eight, at least nine, at least 10, or all 11 cysteine pairs selected from the group consisting of (a) to (k).

Clause 10: The modified VZV gE protein of any one of clauses 8-9 comprising at least one cysteine pair selected from (a) and (b), and at least one cysteine pair selected from (c) to (k).

Clause 11: The modified VZV gE protein of any one of clauses 8-10 comprising the cysteine pairs of (a) and (b), and at least one cysteine pair of (c) to (k).

Clause 12: The modified VZV gE protein of clause 8 comprising the cysteine pair of (a).

Clause 13: The modified VZV gE protein of clause 8 comprising the cysteine pair of (b).

Clause 14: The modified VZV gE protein of clause 8 comprising the cysteine pairs of (a) and (b).

Clause 15: The modified VZV gE protein of clause 8 comprising the cysteine pairs of (a), (b) and (c).

Clause 16: The modified VZV gE protein of clause 8 comprising the cysteine pairs of (a), (b) and (d).

Clause 17: The modified VZV gE protein of clause 8 comprising the cysteine pairs of (a), (b) and (e).

Clause 18: The modified VZV gE protein of clause 8 comprising the cysteine pairs of (a), (b) and (f).

Clause 19: The modified VZV gE protein of clause 8 comprising the cysteine pairs of (a), (b) and (g).

Clause 20: The modified VZV gE protein of clause 8 comprising the cysteine pairs of (a), (b) and (h).

Clause 21: The modified VZV gE protein of clause 8 comprising the cysteine pairs of (a), (b) and (i).

Clause 22: The modified VZV gE protein of clause 8 comprising the cysteine pairs of (a), (b) and (j).

Clause 23: The modified VZV gE protein of clause 8 comprising the cysteine pairs of (a), (b) and (k).

Clause 24: The modified VZV gE protein of clause 8 comprising the cysteine pairs of (a), (b), (c) and (d).

Clause 25: The modified VZV gE protein of clause 8 comprising the cysteine pairs of (a), (b), (c) and (f).

Clause 26: The modified VZV gE protein of clause 8 comprising the cysteine pairs of (a), (b), (c) and (g).

Clause 27: The modified VZV gE protein of clause 8 comprising the cysteine pairs of (a), (b), (d) and (f).

Clause 28: The modified VZV gE protein of clause 8 comprising the cysteine pairs of (a), (b), (c) and (g).

Clause 29a: The modified VZV gE protein of clause 8 comprising the cysteine pairs of (a), (b), (f) and (g).

Clause 29b: The modified VZV gE protein of clause 1 comprising at least one cysteine pair selected from the group consisting of: Cys-142 and Cys-285; Cys-144 and Cys-287; Cys-146 and Cys-152; Cys-146 and Cys-261; Cys-147 and Cys-150; Cys-154 and Cys-256; Cys-156 and Cys-254; Cys-156 and Cys-158; Cys-156 and cys-271; Cys-158 and Cys-254; Cys-161 and Cys-164; Cys-164 and Cys-277; Cys-164 and Cys-275; Cys-165 and Cys-276; Cys-166 and Cys-275; Cys-167 and Cys-219; Cys-167 and Cys-274; Cys-168 and Cys-273; Cys-169 and Cys-217; Cys-169 and Cys-272; Cys-170 and Cys-215; Cys-171 and Cys-214; Cys-171 and Cys-270; Cys-173 and Cys-268; Cys-216 and Cys-251; Cys-217 and Cys-236; Cys-217 and Cys-235; Cys-220 and Cys-232; Cys-238 and Cys-240; Cys-239 and Cys-242; Cys-241 and Cys-248; Cys-241 and Cys-246; Cys-244 and Cys-246; Cys-250 and Cys-257; Cys-269 and Cys-285; Cys-271 and Cys-283; Cys-275 and Cys-277; Cys-275 and Cys-279; and Cys-289 and Cys-292, wherein the cysteines are numbered with respect to SEQ ID NO: 1.

Clause 30: The modified VZV gE protein of any one of clauses 1-29, further comprising at least one cavity-filling mutation in addition to the cysteine pair.

Clause 31: The modified VZV gE protein of clause 30 wherein the at least one cavity-filling mutation is selected from the group consisting of S443Y, S443M, S443F, S443I, L442Y, L442F, L442I, F342H, F342M, F342T, F342A, G429W, G429M, T409M, T409V, T409I, T409L, T409F, A395L, A395V, A395M, A395F, I337M, I337F, I337L; wherein the stabilizing point mutations are numbered with respect to SEQ ID NO: 1.

Clause 32: The modified VZV gE protein of any one of clauses 1 to 31, further comprising a least one combination of stabilizing point mutations selected from the group consisting of: a) T356R, L390W, and Y408W, b) T356S, M379E, S381A, G382K, L390W, Q392L, Q400P, Y408W, T409W, K434V, D437N, and F479L, c) M331V, E339D, A340E, T356R, Q358G, M379E, N380D, S381A, G382R, L390W, Q392L, Q400P, E403T, Y408W, T409W, K434V, D437N, T462I, V467L, and F479L, and d) M331V, L390W, T462I, F479L; wherein the stabilizing point mutations are numbered with respect to SEQ ID NO: 1.

Clause 33: The modified VZV gE protein of any one of clauses 1-32 having an amino acid sequence at least 85%, 86%, 87%, 88%, 89%, 90%, 91%, 92%, 93%, 94%, 95%, 96%, 97%, 98%, or 99% identical to a non-modified VZV gE protein.

Clause 34: The modified VZV gE protein of clause 33 wherein the non-modified VZV gE protein comprises an amino acid sequence selected from the group consisting of SEQ ID NOs: 1-7.

Clause 35: The modified VZV gE protein of any one clauses 33-34 comprising an amino acid sequence at least 85%, 86%, 87%, 88%, 89%, 90%, 91%, 92%, 93%, 94%, 95%, 96%, 97%, 98%, or 99% identical to an amino acid sequence selected from the group consisting of SEQ ID NOs: 10-27.

Clause 36: The modified VZV gE protein of any one clauses 33-34 comprising an amino acid sequence at least 85%, 86%, 87%, 88%, 89%, 90%, 91%, 92%, 93%, 94%, 95%, 96%, 97%, 98%, or 99% identical to an amino acid sequence selected from the group consisting of SEQ ID NOs: 28-45.

Clause 37: The modified VZV gE protein of any one of clauses 1-36, further comprising a signal sequence.

Clause 38: The modified VZV gE protein of clause 37, wherein the signal sequence comprises an amino acid sequence at least 90% identical to SEQ ID NO: 8.

Clause 39: The modified VZV gE protein of any one of clauses 1-38 having improved stability over a non-modified VZV gE protein.

Clause 40: The modified VZV gE protein of any one of clauses 1-38 having a higher melting temperature (Tm) relative to a non-modified VZV gE protein.

Clause 41: The modified VZV gE protein of clause 40 having a Tm at least 2, at least 3, at least 4, at least 5, at least 10, at least 15, at least 16, at least 17, at least 18, at least 19, at least 20, at least 25, or at least 30 degrees C. higher than a non-modified VZV gE protein.

Clause 42: The modified VZV gE protein of any one clauses 39-40 wherein Tm is determined by differential scanning fluorimetry.

Clause 43: The modified VZV gE protein of clauses 1-42 which elicits an immune response against VZV, such as an immune response against VZV gE, when administered to a subject.

Clause 44: The modified VZV gE protein of clause 43, wherein the immune response is an antigen specific B cell response, such as a neutralizing antibody response.

Clause 45: The modified VZV gE protein of any one of clauses 43-44, wherein the immune response is an antigen specific T-cell response, such as a CD4+ T cell response or a CD8+ T cell response.

Clause 46: The modified VZV gE protein of any one of clauses 43-45 wherein the immune response is at least equivalent to the immune response elicited by an equivalent amount of non-modified VZV gE protein.

Clause 47: The modified VZV gE protein of any one of clauses 43-46 wherein the immune response is increased by at least 10%, at least 20%, at least 30%, at least 40%, at least 50%, or at least 100% (twofold) compared to the immune response elicited by an equivalent amount of non-modified VZV gE.

Clause 48: The modified VZV gE protein of any one of clauses 43-47 wherein the immune response has a duration that is at least equivalent to the duration of the immune response elicited by an equivalent amount of non-modified VZV gE protein.

Clause 49: The modified VZV gE protein of clause 48 wherein the duration of the immune response is increased by at least 10%, at least 20%, at least 30%, at least 40%, at least 50%, or at least 100% (twofold) compared to the duration of the immune response elicited by an equivalent amount of non-modified VZV gE.

Clause 50: A nucleic acid encoding the modified VZV gE protein of any one of clauses 1-49.

Clause 51: The nucleic acid of clause 50 which is DNA.

Clause 52: The nucleic acid of clause 50 which is RNA.

Clause 53: The nucleic acid of clause 52 wherein the RNA is non-replicating RNA or self-replicating RNA.

Clause 54: A vector comprising the nucleic acid of any one of clauses 50-53.

Clause 55: A host cell comprising the vector of clause 54.

Clause 56: An immunogenic composition comprising the modified VZV gE protein of any one of clauses 1-49, the nucleic acid of any one of clauses 50-53, or the vector of clause 54.

Clause 57: The immunogenic composition of clause 56, further comprising an adjuvant.

Clause 58: The immunogenic composition of clause 57 wherein the adjuvant comprises a saponin, a TLR4 agonist and liposomes.

Clause 59: The immunogenic composition of clause 58 wherein the saponin is QS21.

Clause 60: The immunogenic composition of clauses 58-59 wherein the TLR4 agonist is 3-O-desacyl-4′-Monophosphoryl Lipid A (3D-MPL).

Clause 61: The immunogenic composition of any one of clauses 56-60 which is a liquid.

Clause 62: The immunogenic composition of any one of clauses 56-60 which is lyophilized.

Clause 63: A kit comprising the immunogenic composition of clause 56 and an adjuvant.

Clause 64: The kit of clause 63, wherein the immunogenic composition is lyophilized and the adjuvant is liquid.

Clause 65: A method of enhancing an immune response to VZV in a subject comprising administering the modified VZV gE protein of any one of clauses 1-49, the nucleic acid of any one of clauses 50-53, the vector of clause 54, or the immunogenic composition of any one of clauses 56-62 to the subject.

Clause 66: A method of preventing or ameliorating herpes zoster (HZ) and/or post herpetic neuralgia (PHN) in a subject comprising administering the modified VZV gE protein of any one of clauses 1-49, the nucleic acid of any one of clauses 50-53, the vector of clause 54, or the immunogenic composition of any one of clauses 56-62 to the subject.

Clause 67: The method of clauses 65-66 wherein the immunogenic composition is administered in a single dose regimen.

Clause 68: The method of clauses 65-66 wherein the VZV gE is administered in a two-dose regimen.

Clause 69: The method of any one of clauses 65-68, wherein the immunogenic composition is administered to an adult subject at a dose of less than 50 mcg of modified VZV gE protein.

Clause 70: The method of any one of clauses 65-69, wherein the immunogenic composition is administered to an adult subject at an adjuvant dose of less than 50 mcg of 3-O-desacyl-4′-Monophosphoryl Lipid A (3D-MPL).

Clause 71: The method of any one of clauses 65-70, wherein the immunogenic composition is administered to an adult subject at a dose of less than 50 mcg of QS21.

Clause 72: The modified VZV gE protein of any one of clauses 1-49, the nucleic acid of any one of clauses 50-53, the vector of clause 54, or the immunogenic composition of any one of clauses 56-62 for use in enhancing an immune response to VZV in a subject.

Clause 73: The modified VZV gE protein of any one of clauses 1-49, the nucleic acid of any one of clauses 50-53, the vector of clause 54, or the immunogenic composition of any one of clauses 56-62 for use in preventing or ameliorating herpes zoster (HZ) and/or post herpetic neuralgia (PHN) in a subject.

Clause 74: Use of the modified VZV gE protein of any one of clauses 1-49, the nucleic acid of any one of clauses 50-53, the vector of clause 54, or the immunogenic composition of any one of clauses 56-62 for enhancing an immune response to VZV in a subject.

Clause 75: Use of the modified VZV gE protein of any one of clauses 1-49, the nucleic acid of any one of clauses 50-53, the vector of clause 54, or the immunogenic composition of any one of clauses 56-62 for preventing or ameliorating herpes zoster (HZ) and/or post herpetic neuralgia (PHN) in a subject.

### EXAMPLES

Many modifications and variations of the present disclosure are possible in light of the above teachings. It is therefore to be understood that, within the scope of the appended claims, a skilled person in the art would recognize that the invention may be practiced otherwise than as specifically described. The illustrative embodiments and examples should not be construed as limiting the invention.

### Example 1: Thermostability of Vzv Ge Protein

To gauge the stability enhancement potential for modified VZV gE proteins, thermostability was assessed for the full wild-type VZV gE ectodomain (“Full length gE” corresponding to amino acids 1 to 507 of SEQ ID NO: 1) and a subcleaved fragment (“subcleaved gE”) corresponding to amino acids 116 to 516 of SEQ ID NO: 1) lacking the N-terminal 115 amino acids of VZV gE ectodomain.

Expression vectors encoding the full length VZV gE ectodomain (amino acids 1 to 507 of SEQ ID NO: 1) or the subcleaved fragment (amino acids 116 to 516 of SEQ ID NO: 1) were prepared using standard methods. The vectors further encoded an N-terminal signal peptide to facilitate expression (SEQ ID NO: 8), and a C-terminal histidine tag to allow purification (SEQ ID NO: 9).

Expression vectors were transfected into cells using the Gibco Expi293 Expression System (ThermoFisher) according to the manufacturer's protocol. Briefly, Expi293 cell cultures (3×100 mL) were transfected with DNA from each construct. Protein was harvested on day 5 post-transfection. The harvested protein was purified by Nickel affinity chromatography. Pooled peaks were subjected to size exclusion chromatography (SEC).

Samples of purified full length and subcleaved gE were diluted to a concentration of 1 mg/mL in buffer containing 10 mM Tris pH 7.5 and 150 mM NaCl. Melting temperature (Tm) of each sample was assessed using nano-differential scanning fluorimetry (nDSF) on a Nanotemper Prometheus NT.48 instrument. The intrinsic fluorescence of aromatic residues, such as Tyr and Trp, was obtained by exciting at 280 nm wavelength and measuring emission spectra at 330 nm (representing the folded state) and 350 nm (representing the unfolded state) over a temperature ramp (25° C. to 90° C.), which was increased at a rate of 1° C. per minute. The instrument software was used to plot the differential of the fluorescence ratio (350 nm/330 nm), and the temperature corresponding to the inflection point of the curve was taken as the melting temperature of the sample protein (Tm).

The experiment was carried out in triplicate and Tm was calculated as the average of each set of measurements per sample. The Tm for both full length VZV gE ectodomain and the subcleaved fragment were determined to be 53° C. The results support the prediction that the N-terminus of wild-type VZV gE ectodomain is likely disordered and does not contribute to the thermostability of the full ectodomain.

### Example 2: Crystal Structure of Vzv Ge Protein

To facilitate the design of more thermostable VZV gE proteins, the present inventors undertook to solve the 3-dimensional structure of the wild-type VZV gE ectodomain. Due to the inherent flexibility of the N-terminus of the full length ectodomain, crystal structures were solved for two major subdomains of VZV gE: the Glycoprotein I Binding Domain (gIBD) and the Fc Binding Domain (FcBD).

1. Structure of the VZV gE gIBD

An expression vector encoding a VZV gE ectodomain fragment (amino acids 116 to 305 of SEQ ID NO: 1) was prepared using standard methods. The fragment was selected to encompass the VZV gE glycoprotein I Binding Domain (gIBD). The vector further encoded an N-terminal signal peptide to facilitate expression (SEQ ID NO: 8), and a TEV cleavage site and C-terminal histidine tag to allow purification (SEQ ID NO: 9).

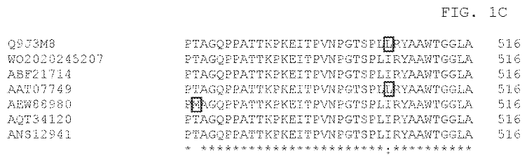

When the gIBD expression vector was transfected into Expi293 cells alone, the gIBD protein failed to express, or was aggregated thus preventing purification. However, the construct could be successfully expressed and purified from Expi293 cells when co-expressed with fragment antigen binding region (Fab) of anti-gE antibodies (such as Fab 1E3, Sullivan et. al, 2018). The expressed gIBD protein/Fab complexes were then partially purified using Nickel affinity chromatography. The complexes were further purified by size exclusion chromatography (SEC) in buffer containing 10 mM Tris pH 7.5 and 150 mM NaCl. SDS-PAGE and Western blot analysis confirmed the presence of the gIBD/Fab complexes (FIG. 2). Expression of the complex was further confirmed by HPLC (not shown).

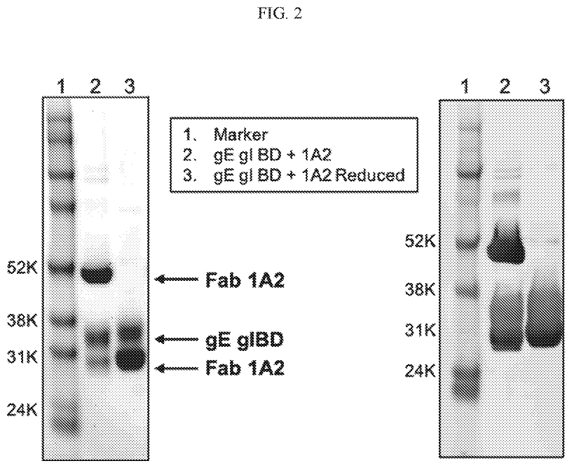

The gIBD-Fab1E3 complex was crystallized in buffer containing 0.2M ammonium sulfate, 20% PEG 3350) and the structure was solved to a resolution of 2.3 Å (FIG. 3). Visible portions of the structure are from residues Leu141-Glu293, with residues from Pro174 through Ala216 being flexible and not present in the structure. This loop (specifically residues 178-206) is annotated as the gI binding site (https://www.uniprot.org/uniprot/P09259).

### 2. Structure of the VZV gE FcBD

An expression vector encoding a VZV gE ectodomain fragment (SEQ ID NO: 46) was prepared using standard methods. The fragment was designed to encompass the VZV gE Fc Binding Domain (FcBD). The vector further encoded an N-terminal signal peptide to facilitate expression (SEQ ID NO: 8), and a C-terminal histidine tag to allow purification (SEQ ID NO: 9).

An expression vector encoding Fab 5A2 was prepared using standard methods and expressed in Expi293 cells. Fab 5A2 was partially purified using Nickel affinity chromatography and further purified by size exclusion chromatography (SEC) in buffer containing 10 mM Tris pH 7.5 and 150 mM NaCl. FcBD was incubated with excess Fab 5A2 overnight and then the FcBD-Fab 5A2 complex purified by SEC into the same buffer.

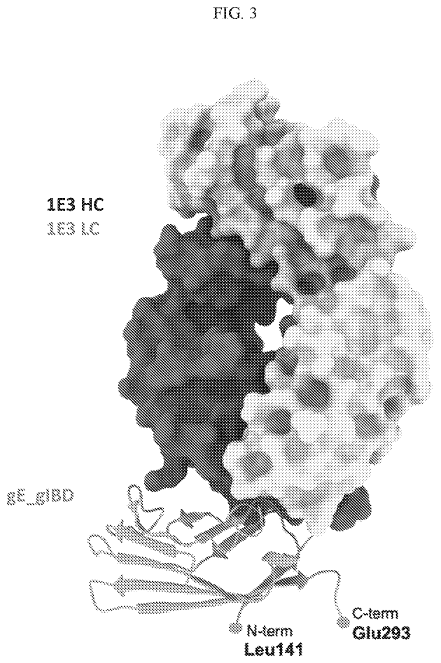

FcBD-Fab5A2 was crystallized in buffer containing 2.0 M Ammonium sulfate, 0.1M Bis-Tris pH 6.5 and additive benzamidine hydrochloride and the x-ray structure solved at a resolution of 4.1 Å (FIG. 4).

### Example 3: Point Mutations

By analyzing the crystal structures elucidated in Example 2, the present inventors identified cavities within the VZV gE ectodomain structure which could contribute to protein instability. Cavity-filling mutations in the Fc Binding Domain (FcBD) were designed and the impact of these mutations on thermostability was assessed.

The Molecular Operating Environment software platform (MOE, 2020.09 Chemical Computing Group ULC, 1010 Sherbooke St. West, Suite #910, Montreal, QC, Canada, H3A 2R7, 2022) was used to identify putative point mutations in the gE Fc binding domain (FcBD) which could lead to increased thermostability. The crystal structure of VZV gE216 (FcBD) identified in Example 2 was used with the energy minimized using default settings (quick prep). Using the Protein Design feature, a Sequence Design run was performed in which every amino acid was mutated to each of the 20 amino acids and the dStability calculated. Seven residues were predicted to be susceptible to stabilizing mutations (dStability=negative; F342, T432, G429, L442, H317, S443, and H338), for which the top four (S443, F342, L442, and G429) were each selected and between 2 to four mutations per site introduced, for a total of 13 individual point mutations (Table 6).

Overall, these point mutants did not result in any mutant having a Tm higher than WT (53° C.).

TABLE 6
Cavity-filling mutations in gE FcBD

| Mutations relative | | |
|---|---|---|
| to WT gE | | |
| (SEQ ID NO: 1) | dStablility | Tm, ° C. |
| S443Y | −0.3 | 51 |
| S443M | −0.29 | 52 |
| S443F | −0.22 | 52 |
| S443I | −0.31 | 52.5 |
| L442Y | −0.5 | 53 |
| L442F | −0.72 | 53 |
| L442I | −1.3 | 51.3 |
| F342H | −1.6 | Did not express |
| F342M | −2.2 | 50.4 |
| F342T | −1.4 | Inconclusive DSF results |
| F342A | −1.1 | 50.4 |
| G429W | −0.7 | 51.8 |
| G429M | −0.45 | 51 |

### Rational Design of Cavity Filling Mutants

Pymol visualization software was used to identify cavities within the gE216 (FcBD) structure (surface display of cavities with 4 Angstrom radius). These cavities were visually inspected to identify surrounding residue(s) which could be mutated in order to fill the cavity and lead to greater thermostability. Four cavities (or pockets) were identified as candidates for mutagenesis of surrounding residues.
Pocket 1—mutation of T409
Pocket 2—Mutation of 1337
Pocket 3—Mutation of A391
Pocket 4—Mutation of A395

Five mutations of T409, four mutations of A395, and 3 mutations of 1337 (all numbered according to SEQ ID NO: 1) were engineered in order to comprehensively evaluate the potential for larger residues to fill nearby pockets and improve thermostability (Table 7); however, all mutants engineered into VZV gE showed similar or lower melting temperature (Tm) compared to wild-type VZV gE, suggesting that cavity filling mutations are not an optimal approach for stabilizing gE.

TABLE 7
Cavity-filling mutations in gE FcBD

| Mutations relative | |
|---|---|
| to WT gE | |
| (SEQ ID NO: 1) | Tm, ° C. |
| T409M | 52.9 |
| T409V | 52.7 |
| T409I | 52.1 |
| T409L | 51.9 |
| T409F | 51.9 |
| A395L | 50.3 |
| A395V | 50.8 |
| A395M | 49.3 |
| A395F | 50.1 |
| I337M | 50 |
| I337F | 50 |
| I337L | 50 |

### Example 4: Evolutionary in Silico Folding Mutations

Evolutionary Sequence Alignment: Five hundred homologous gE sequences were obtained from the nonredundant BLAST database. These aligned sequences were calculated into a position-specific scoring matrix (PSSM) with the PSI-BLAST algorithm. The matrix represents the likelihood of the 20 amino acids being present at each residue position, within the aligned sequences.

Minimization and Residue Mutant Scanning: The C-terminal gE x-ray structure was minimized in YASARA and Rosetta. The Rosetta FilterScan mover was used to perform single point mutagenesis of all the residues to the preferred PSSM mutations. The mutation scan was binned within nine different energy thresholds (0.5, −0.45, −0.75, −1, −1.25, −1.5, −1.8, −2, −3 kcal/mol) to increase mutation sequence diversity. For example, a combination of −2 kcal/mol single point mutations would result in fewer mutations due to a higher energetic barrier for introducing new mutations.

Combinatorial Design: A RosettaScripts algorithm that energetically combined the proposed single mutations was used to reduce the search space, yielding nine total stabilizing designs, representing each energy threshold. Three of the designs were chosen for in vitro testing (gE-neg3, gE-neg1.5, gE-neg075) along with one design (gE-WH) which was a visual “chimera” of mutations across each of the nine computationally designed constructs.

Results are summarized in Table 8. Designs gE neg075 and neg3 did not express in high enough levels to test the thermal melting temperature, while designs gE-neg 1.5 and gE-WH each expressed at similar yield as wild type gE, although with Tm values either similar (within 1 degree c.) or lower than wild-type VZV gE.

TABLE 8
Consensus Sequence Designs.

| | | Construct | | |
|---|---|---|---|---|
| Wt | | Naming | Expression | deltaTm, |
| Residue(s) | Mutation(s) | Convention | (+, −) | ° C. |
| 356, 390, | T356R, L390W, | gE_neg3 | − | N/A |
| 408 | Y408W | | | |
| 356, 379, | T356S, M379E, | gE_neg1.5 | + | 0.8 |
| 381, 382, | S381A, G382K, | | | |
| 390, 392, | L390W, Q392L, | | | |
| 400, 408, | Q400P, Y408W, | | | |
| 409, 434, | T409W, K434V, | | | |
| 437, 479 | D437N, F479L | | | |
| 331, 339, | M331V, E339D, | gE_neg075 | − | N/A |
| 340, 356, | A340E, T356R, | | | |
| 358, 379, | Q358G, M379E, | | | |
| 380, 381, | N380D, S381A, | | | |
| 382, 390, | G382R, L390W, | | | |
| 392, 400, | Q392L, Q400P, | | | |
| 403, 408, | E403T, Y408W, | | | |
| 409, 434, | T409W, K434V, | | | |
| 437, 462, | D437N, T462I, | | | |
| 467, 479 | V467L, F479L | | | |
| 331, 390, | M331V, L390W, | gE_WH | + | −2.6 |
| 462, 479 | T462I, F479L | | | |

### Example 5: Disulfide Bridge Stabilization of Fc Binding Domain

Analysis of the VZV gE FcBD crystal structure in complex with Fab 5A2 revealed the following candidate sites for the introduction of stabilizing non-native disulfide-bridging cysteines in VZV gE (amino acids numbered with respect to SEQ ID NO: 1):
H-427 and K-434 each substituted with cysteine, as exemplified by SEQ ID Nos: 10 and 28
T-365 and R-477 each substituted with cysteine, as exemplified by SEQ ID Nos: 11 and 29

Modified VZV gE proteins containing either or both of the non-native disulfide bridges in the FcBD were expressed and characterized as summarized in Table 9:

TABLE 9
Modified VZV gE proteins with non-
native disulfide bridges in FcBD

| Modified gE | SEQ ID | |
|---|---|---|
| Design No. | NO: | Description |
| Des-1 | 10 | WT VZV gE ectodomain of SEQ ID NO: 1 with |
| | | a disulfide bridge introduced by mutations |
| | | H427C and K434C |
| Des-2 | 11 | WT VZV gE ectodomain of SEQ ID NO: 1 with |
| | | a disulfide bridge introduced by mutations |
| | | T365C and R477C |
| Des-3 | 12 | WT VZV gE ectodomain of SEQ ID NO: 1 with |
| | | the disulfide bridges of Des-1 and Des-2: |
| | | H427C and K434C; T365C and R477C |

Each of the three modified VZV gE proteins was expressed in Expi293 cells from vectors encoding an N-terminal signal peptide (SEQ ID NO: 8) and a C-terminal histidine tag (SEQ ID NO: 9). The modified VZV gE proteins were harvested and purified by Nickel affinity chromatography and SEC as described elsewhere herein.

The nanoDSF assay was carried out in buffer containing 10 mM Tris pH 7.5, 150 mM NaCl, with each protein at a concentration of 1 mg/mL and wild-type VZV gE as a control. Melting temperature (Tm) values were calculated as the average of the 1st derivative for each set of triplicate measurements per sample.

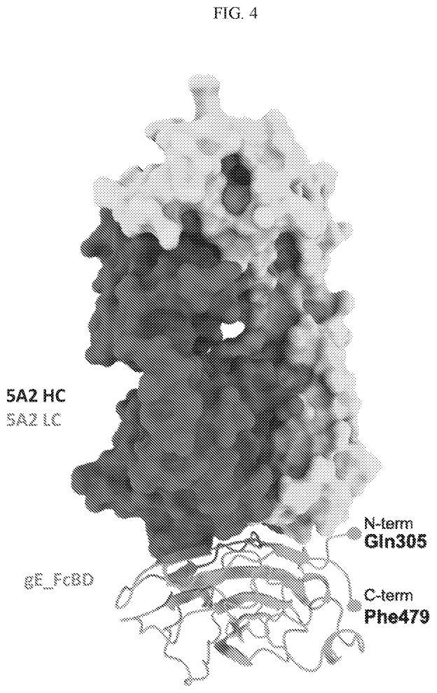

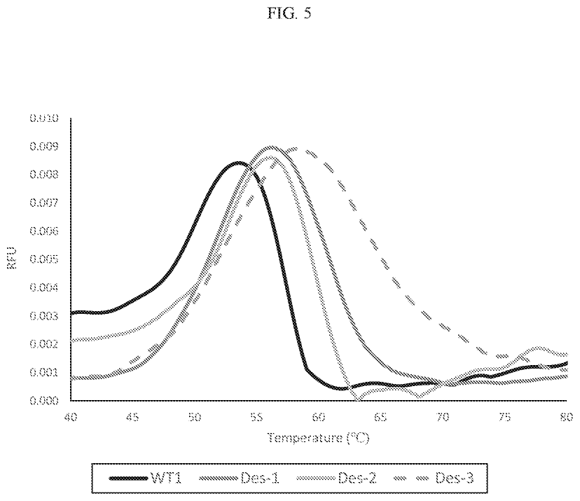

As shown in FIG. 5, the single disulfide bridges (Des-1 and Des-2) each produced a 3° C. increase in Tm relative to wild-type VZV gE, as measured by nanoDSF. The combination of both disulfide bridges (Des-3) produced an increase in Tm of 5° C. as measured by nanoDSF.

### Example 6: Disulfide Bridge Stabilization of Gi Binding Domain

The VZV gE gIBD crystal structure in complex with Fab 1E3 was used to calculate feasible sites for the introduction of non-native disulfide-bridges. The following positions were identified for the introduction of cysteine pairs capable of forming non-native disulfide bridges (amino acids numbered with respect to SEQ ID NO: 1):
A-216 and 1-251 each substituted with cysteine, as exemplified by SEQ ID Nos: 13 and 31
1-158 and G-254 each substituted with cysteine, as exemplified by SEQ ID Nos: 14 and 32
I-161 and V-164 each substituted with cysteine, as exemplified by SEQ ID Nos: 15 and 33
S-171 and Q-214 each substituted with cysteine, as exemplified by SEQ ID Nos: 16 and 34
T-167 and S-219 each substituted with cysteine, as exemplified by SEQ ID Nos: 17 and 35
T-169 and E-217 each substituted with cysteine, as exemplified by SEQ ID Nos: 18 and 36
V-144 and V-287 each substituted with cysteine, as exemplified by SEQ ID Nos: 19 and 37
V-164 and D-277 each substituted with cysteine, as exemplified by SEQ ID Nos: 20 and 38
Y-220 and W-232 each substituted with cysteine, as exemplified by SEQ ID Nos: 21 and 39

Modified VZV gE proteins containing non-native disulfide bridges in the FcBD combined with non-native disulfide bridges in the gIBD (from Example 5) were expressed and characterized, as summarized in Table 10.

Each of the modified VZV gE proteins was expressed in Expi293 cells from vectors encoding an N-terminal signal peptide (SEQ ID NO: 8) and a C-terminal histidine tag (SEQ ID NO: 9). The modified VZV gE proteins were harvested and purified by Nickel affinity chromatography and SEC as described elsewhere herein.

The nanoDSF assay was carried out in buffer containing 10 mM Tris pH 7.5, 150 mM NaCl, with each protein at a concentration of 1 mg/mL and wild-type VZV gE as a control. Melting temperature (Tm) values were calculated as the average of the 1st derivative for each set of triplicate measurements per sample.

The results are summarized in Table 10 below. All six modified VZV gE proteins tested had higher melting temperatures than wild-type VZV gE (52.9° C.). One construct (Des-5) exhibited a Tm increase of about 20° C. compared to wild-type gE.

TABLE 10

| Modified gE | SEQ | | Tm |
|---|---|---|---|
| Design | ID NO | Description | ° C. |
| WT1 VZV gE | 1 | Wild-type 1 (WT1) VZV gE ectodomain of Genbank | 52.9 |
| | | Accession No. Q9J3M8 | |
| Des-4 | 13 | WT1 VZV gE ectodomain of SEQ ID NO: 1 with | 70.5 |
| | | disulfide bridges introduced by mutations H427C, K434C; | |
| | | T365C, R477C; A216C, I251C | |
| Des-5 | 14 | WT1 VZV gE ectodomain of SEQ ID NO: 1 with | 72.8 |
| | | disulfide bridges introduced by mutations H427C, K434C; | |
| | | T365C, R477C; I158C, G254C | |
| Des-7 | 16 | WT1 VZV gE ectodomain of SEQ ID NO: 1 with | 63.3 |
| | | disulfide bridges introduced by mutations H427C, K434C; | |
| | | T365C, R477C; S171C, Q214C | |
| Des-8 | 17 | WT1 VZV gE ectodomain of SEQ ID NO: 1 with | 69.4 |
| | | disulfide bridges introduced by mutations H427C, K434C; | |
| | | T365C, R477C; T167C, S219C | |
| Des-9 | 18 | WT1 VZV gE ectodomain of SEQ ID NO: 1 with | 70.3 |
| | | disulfide bridges introduced by mutations H427C, K434C; | |
| | | T365C, R477C; T169C, E217C | |
| Des-11 | 20 | WT VZV gE ectodomain of SEQ ID NO: 1 with disulfide | 63.3 |
| | | bridges introduced by mutations H427C, K434C; T365C, | |
| | | R477C; V164C, D277C | |

### Example 7: Disulfide Bridge Stabilization of Vzv Ge Wild-Type Variant

The experiments described in Examples 1-6 were conducted using the wild-type VZV gE ectodomain sequence (“WT1”) of Genbank Accession Q9J3M8 (SEQ ID NO: 1). To demonstrate that other gE WT variants can be stabilized by the same mutations, modified VZV gE proteins of Example 6 were prepared using the wild-type VZV gE sequence of SEQ ID NO: 2 (“WT2”). The VZV gE variant of SEQ ID NO: 2 differs from SEQ ID NO: 1 in three positions: amino acid 120 (D/N), amino acid 466 (T/Y) and amino acid 506 (L/I).

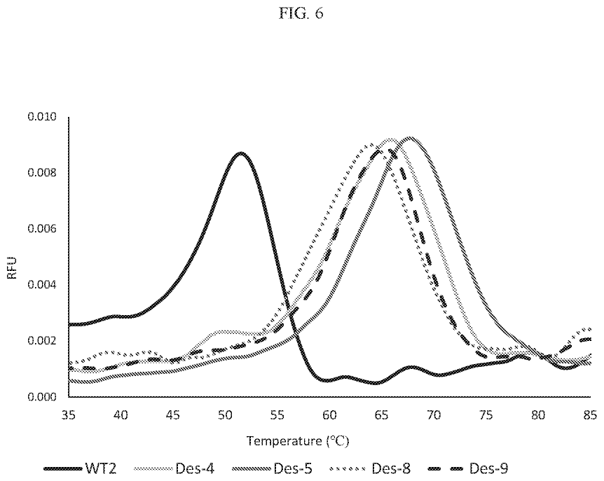

Modified VZV gE proteins were expressed and characterized by nanoDSF as described in Example 6. The results are summarized in Table 11 below. The melting temperature curves are shown in FIG. 6. Tm is presented as the average of triplicate assays. All six modified VZV gE proteins assayed had higher melting temperatures than wild-type VZV gE (51.4° C.). As observed in Example 6, Des-5 was the most thermostable design tested.

TABLE 11

| Modified gE | SEQ | | Tm |
|---|---|---|---|
| Design | ID NO | Description | (° C.) |
| WT2 VZV gE | 2 | Wild-type 2 (WT2) VZV gE ectodomain of PCT Publication | 51.4 |
| | | WO2020245207 (sequence 6) | |
| Des-4 | 31 | WT2 VZV gE ectodomain of SEQ ID NO: 1 with disulfide | 65.6 |
| | | bridges introduced by mutations H427C, K434C; T365C, | |
| | | R477C; A216C, I251C | |
| Des-5 | 32 | WT2 VZV gE ectodomain of SEQ ID NO: 1 with disulfide | 67.7 |
| | | bridges introduced by mutations H427C, K434C; T365C, | |
| | | R477C; I158C, G254C | |
| Des-8 | 35 | WT2 VZV gE ectodomain of SEQ ID NO: 1 with disulfide | 63.8 |
| | | bridges introduced by mutations H427C, K434C; T365C, | |
| | | R477C; T167C, S219C | |
| Des-9 | 36 | WT2 VZV gE ectodomain of SEQ ID NO: 1 with disulfide | 65.0 |
| | | bridges introduced by mutations H427C, K434C; T365C, | |
| | | R477C; T169C, E217C | |

### Example 8: Combinations of Four Non-Native Disulfide Bridges

Additional combinations of the modified VZV gE designs characterized in Examples 5-7 were made and characterized to determine if further improvement to VZV gE thermostability could be achieved by introducing four non-native disulfide bridges into the VZV gE ectodomain.

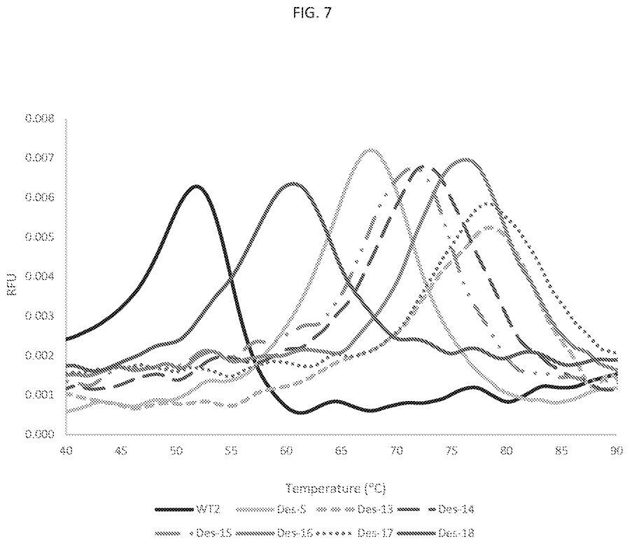

Modified VZV gE proteins were expressed and characterized by nanoDSF as described in Example 6. The results are summarized in Table 12 below. The melting temperature curves are shown in FIG. 7. Tm is presented as the average of triplicate assays. All modified VZV gE proteins assayed had higher melting temperatures than wild-type VZV gE. Des-13 and Des-17 exhibited melting temperatures 26.8° C. higher than wild-type VZV gE.

TABLE 12

| Modified gE | SEQ | | Tm |
|---|---|---|---|
| Design | ID NO | Description | (° C.) |
| WT2 VZV gE | 2 | Wild-type 2 (WT2) VZV gE ectodomain of PCT Publication | 51.6 |
| | | WO2020245207 (sequence 6) | |
| Des-5 | 32 | as described in Example 7 (included for comparison) | 67.7 |
| Des-13 | 40 | Combination of Des-4 and Des-5, having disulfide bridges | 78.4 |
| | | introduced by mutations H427C, K434C; T365C, R477C; | |
| | | A216C, I251C; I158C, G254C | |
| Des-14 | 41 | Combination of Des-4 and Des-8, having disulfide bridges | 72.7 |
| | | introduced by mutations H427C, K434C; T365C, R477C; | |
| | | A216C, I251C; T167C, S219C | |
| Des-15 | 42 | Combination of Des-4 and Des-9, having disulfide bridges | 71.3 |
| | | introduced by mutations H427C, K434C; T365C, R477C; | |
| | | A216C, I251C; T169C, E217C | |
| Des-16 | 43 | Combination of Des-5 and Des-8, having disulfide bridges | 76.1 |
| | | introduced by mutations H427C, K434C; T365C, R477C; | |
| | | I158C, G254C; T167C, S219C | |
| Des-17 | 44 | Combination of Des-5 and Des-9, having disulfide bridges | 78.4 |
| | | introduced by mutations H427C, K434C; T365C, R477C; I158C, | |
| | | G254C; T169C, E217C | |
| Des-18 | 45 | Combination of Des-8 and Des-9, having disulfide bridges | 60.5 |
| | | introduced by mutations H427C, K434C; T365C, R477C; | |
| | | T167C/S219C; T169C, E217C | |

### Example 9: In Vitro Binding to Neutralizing Vzv 2E Antibodies

Using a ForteBio Octet96 Red instrument, bio-layer interferometry was conducted to confirm that VZV gE antibodies still bind to the stabilizing mutant designs. mAb 5A2 is a neutralizing antibody targeting the FcBD of VZV gE (source citation). mAbs 1A2 and 1E3 are neutralizing antibodies targeting the gIBD of VZV gE (source citation). Fortebio Anti-Human Fab-CH1 2nd Generation (Fab2G) Dip and Read Biosensors were used to capture antibodies. Antibodies were diluted to a concentration of 0.01 mg/mL in a buffer comprised of 1×PBS and 1% Albumin, Bovine (BSA). Three gE constructs were tested at seven different concentrations each, starting at 100 nM and diluted 1:2 each time, also in 1×PBS, 1% BSA. KD values were calculated using the corresponding Octet BLI Analysis 12.2.1.3 software. Results are presented in Table 13 below. In summary, modified VZV gE proteins retained the ability to bind to known anti-gE antibodies despite the presence of non-native disulfide bridges.

TABLE 13
Antibody binding kinetics of wild-type gE (WT2, SEQ ID
NO: 2) and modified VZV gE proteins (Des-13, SEQ ID NO:
40; and Des-18, SEQ ID NO: 45) to antibodies targeting
either the FcBD (mAb 5A2) or gIBD (mAbs 1A2 and 1E3).

| gE construct | Antibody | KD (nM) |
|---|---|---|
| WT2 | 1A2 | 18 |
| Des-13 | 1A2 | 7 |
| Des-18 | 1A2 | 18 |
| WT2 | 1E3 | 7 |
| Des-13 | 1E3 | 4 |
| Des-18 | 1E3 | 6 |
| WT2 | 5A2 | 21 |
| Des-13 | 5A2 | 23 |
| Des-18 | 5A2 | 28 |

### Example 10: Additional Stabilizing Non-Native Disulfide Bridges

The 3D structures of VZV gE gI Binding Domain and Fc Binding Domain obtained in Example 2 were used to identify additional amino acid pairs suitable for substitution with cysteines to introduce stabilizing disulfide bridges. Briefly, residues that had a Cβ-Cβ distance of 5 Å or fewer were identified using the Molecular Operating Environment (MOE) software. These residues are a favorable distance to form a stabilizing disulfide bond.

Modified VZV gE proteins containing one or more of the mutation pairs listed in Table 14 will exhibit increased stability, such as increased thermostability; increase shelf-life; and/or improved immune response(s) when administered to a subject.

TABLE 14
Additional Stabilizing Mutations (numbered relative to SEQ
ID NO: 1 or corresponding positions of SEQ ID NOs: 2-7).
Stabilizing Mutations

I142:F285
V144:V287
V146:F152
V146:T261
E147:H150
L154:L256
A156:G254
A156:I158
A156:W271
I158:G254
I161:V164
V164:D277
V164:G275
R165:S276
Y166:G275
T167:S219
T167:R274
E168:M273
T169:E217
T169:N272
W170:L215
S171:Q214
S171:I270
L173:V268
A216:I251
E217:N236
E217:V235
Y220:W232
S238:L240
T239:D242
F241:P248
F241:L246
L244:L246
E250:K257
Y269:F285
W271:A283
G275:D277
G275:T279
W289:D292

### Example 11: In Vivo Immunogenicity of Modified Vzv Ge Proteins

An in vivo study was performed to assess the immunogenicity of thermostabilized designs Design 5 (Des-5), Design-13 (Des-13), and Design-18 (Des-18) in mice.

All antigens were expressed and purified in Expi293 cells. Purified protein was diluted 1:1 with AS01 adjuvant (liposomal formulation of plant extract Quillaja saponaria Molina, fraction 21 (QS-21), and 3-O-desacyl-4′-monophosphoryl lipid A (MPL) from Salmonella minnesota) to arrive at the final dose. Formulation was performed at room temperature within 1 hour of dosing.

BALB/c female mice (n=9 per condition) were immunized on day 0 and day 21 with 0.5 μg or 2 μg of Des-5, Des-13, Des-18, or wild type gE antigen formulated in AS01 adjuvant.

Following immunization, blood samples were collected on day 35. Samples sat at room temperature for a minimum of 30 minutes, then were centrifuged prior to transfer and storage at −80° C. Samples were analyzed for total anti-gE IgG.

### IgG Binding Assay:

Samples were prepared by taking donor serum and isolating cells. Cells were then activated by incubation with an anti-CD28 and anti-CD49d co-stimulation mixture for 2 hours at 37° C. and 5% CO2.

An ELISA based assay was performed to assess the anti-gE IgG titers. Plates were coated with wild type gE control coating antigen and incubated overnight at 2-8° C. After discarding the coating antigen solution, blocking solution was added and incubated at 37° C. for 1 hour. Following incubation, plates were washed four times. A two-fold dilution was performed across the plate with either gE standards, internal control, or samples. Plates were then incubated shaking at 37° C. The plate was washed four times with wash buffer prior to addition of secondary antibody in 1×PBS, 1% BSA, 0.1% Tween 20. Horse radish peroxidase was added to each well and then plates were incubated for 30 minutes at 37° C. Plates were again washed with before addition of freshly made O-Phenylenediamine (OPD). Samples were incubated for 20 minutes at room temperature away from light. After OPD development, Stop Solution was then added to each well. Plates were read in a plate reader between 490-620 nm. Titers were calculated using a 4PL curve.

### Statistics:

An ANOVA model with antigen design, dose, and the antigen design x dose interaction as factors was fitted onto log 10 transformed data. Variance was considered homogeneous across vaccines but was considered heterogeneous across doses. No correction for multiplicity of comparisons was applied. The negative control group (saline group) data was not included in this analysis. From this model, the geometric mean titer (GMT) with a 95% confidence interval (CI) by group was computed. The antigen designs were compared by dose level using the geometric mean ratio (GMR) with a 90% confidence interval to assess non-inferiority of the designs tested.

### Results:

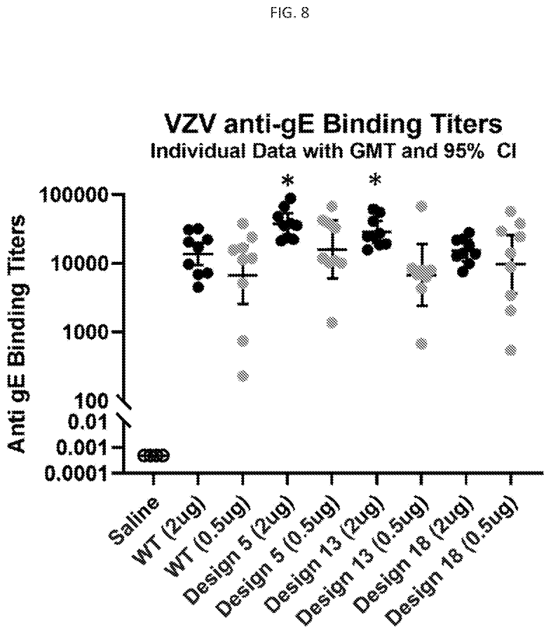

Following two immunizations, all three modified VZV gE designs elicited dose-dependent immune responses as measured by anti-gE IgG levels (FIG. 8). The 2 ug dose of Designs 5 and 13 induced significantly (2.1-2.7 fold) higher immune responses compared to the same dose of the wild-type antigen. The immune response to Design 18 was comparable to that of wild-type gE. All modified antigen designs exhibited lower subject-to-subject variability in the immune response than was observed with wild-type antigen.

These results indicate that cysteine-stabilized gE proteins elicit comparable or higher gE antibody levels, with lower subject-to-subject variability, as compared to wild-type antigen.

### Explanation of Sequences

Amino acid sequences written in N-terminus to C-terminus direction:

Genbank Accession No. Q9J3M8.1 (WT1)
>SEQ ID NO: 1
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSTCLYHPNAPQCLSHMNSGCTFTSPHLAQRVASTVYQNCEHADNY
TAYCLGISHMEPSFGLILHDGGTTLKFVDTPESLSGLYVFVVYENGHVEAVAYTVVSTVDHFVNAIEE
RGFPPTAGQPPATTKPKEITPVNPGTSPLLRYAAWTGGLA
Genbank Accession No. WO2020245207 (WT2)
>SEQ ID NO: 2
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSTCLYHPNAPQCLSHMNSGCTFTSPHLAQRVASTVYQNCEHADNY
TAYCLGISHMEPSFGLILHDGGTTLKFVDTPESLSGLYVFVVYFNGHVEAVAYTVVSYVDHFVNAIEE
RGFPPTAGQPPATTKPKEITPVNPGTSPLIRYAAWTGGLA
Genbank Accession No. ABF21714
>SEQ ID NO: 3
SVLRYDDFHTDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSTCLYHPNAPQCLSHMNSGCTFTSPHLAQRVASTVYQNCEHADNY
TAYCLGISHMEPSFGLILHDGGTTLKFVDTPESLSGLYVFVVYENGHVEAVAYTVVSTVDHFVNAIEE
RGFPPTAGQPPATTKPKEITPVNPGTSPLIRYAAWTGGLA
Genbank Accession No. AAT07749
>SEQ ID NO: 4
SVLRYDDFHTDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSTCLYHPNAPQCLSHMNSGCTFTSPHLAQRVASTVYQNCEHADNY
TAYCLGISHMEPSFGLILHDGGTTLKFVDTPESLSGLYVFVVYFNGHVEAVAYTVVSTVDHFVNAIEE
RGFPPTAGQPPATTKPKEITPVNPGTSPLLRYAAWTGGLA
Genbank Accession No. AEW88980
>SEQ ID NO: 5
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSTCLYHPNAPQCLSHMNSGCTFTSPHLAQRVASTVYQNCEHADNY
TAYCLGISHMEPSFGLILHDGGTTLKFVDTPESLSGLYVFVVYFNGHVEAVAYTVVSTVDHFVNAIEE
RGFPPMAGQPPATTKPKEITPVNPGTSPLIRYAAWTGGLA
Genbank Accession No. AQT34120
>SEQ ID NO: 6
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSTCLYHPNAPQCLSHMNSGCTFTSPHLAQRVASTVYQNCEHADNY
TAYCLGISHMEPSFGLILHDGGTTLKFVDTPESLSGLYVFVVYENGHVEAVAYTVVSTVDHFVNAIEE
RGFPPTAGQPPATTKPKEITPVNPGTSPLIRYAAWTGGLA
Genbank Accession No. ANS12941
>SEQ ID NO: 7
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRVPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSTCLYHPNAPQCLSHMNSGCTFTSPHLAQRVASTVYQNCEHADNY
TAYCLGISHMEPSFGLILHDGGTTLKFVDTPESLSGLYVFVVYFNGHVEAVAYTVVSTVDHFVNAIEE
RGFPPTAGQPPATTKPKEITPVNPGTSPLIRYAAWTGGLA
signal sequence
>SEQ ID NO: 8
MGTVNKPVVGVLMGFGIITGTLRITNPVRA
6-His tag
>SEQ ID NO: 9
GSHHHHHH
Des1_WT1
>SEQ ID NO: 10
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSTCLYHPNAPQCLSHMNSGCTFTSPHLAQRVASTVYQNCEHADNY
TAYCLGISHMEPSFGLILCDGGTTLCFVDTPESLSGLYVFVVYENGHVEAVAYTVVSTVDHFVNAIEE
RGFPPTAGQPPATTKPKEITPVNPGTSPLLR
Des2_WT1
>SEQ ID NO: 11
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILHDGGTTLKFVDTPESLSGLYVFVVYFNGHVEAVAYTVVSTVDHFVNAIEE
CGFPPTAGQPPATTKPKEITPVNPGTSPLLR
Des 3_WT1
>SEQ ID NO: 12
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAFFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYFNGHVEAVAYTVVSTVDHFVNAIEE
CGFPPTAGQPPATTKPKEITPVNPGTSPLLR
Des4_WT1
>SEQ ID NO: 13
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLCEISYRFQGKKEADQPWIVVNTSTLFDELELDPPECEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYFNGHVEAVAYTVVSTVDHFVNAIEE
CGFPPTAGQPPATTKPKEITPVNPGTSPLLR
Des5_WT1
>SEQ ID NO: 14
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPCQRIYGVRYTETWSFLPSLTCTGDAAPAIQHICLKHTTCFQDVVVDV
DCAENTKEDQLAEISYRFQGKKEADQPWIVVNTSTLFDELELDPPEIEPCVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYFNGHVEAVAYTVVSTVDHFVNAIEE
CGFPPTAGQPPATTKPKEITPVNPGTSPLLR
Des6_WT1
>SEQ ID NO: 15
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRCYGCRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYFNGHVEAVAYTVVSTVDHFVNAIEE
CGFPPTAGQPPATTKPKEITPVNPGTSPLLR
Des7_WT1
>SEQ ID NO: 16
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGVRYTETWCFLPSLTCTGDAAPAIQHICLKHTTCFQDVVVDV
DCAENTKEDCLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYENGHVEAVAYTVVSTVDHFVNAIEE
CGFPPTAGQPPATTKPKEITPVNPGTSPLLR
Des 8_WT1
>SEQ ID NO: 17
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGVRYCETWSFLPSLTCTGDAAPAIQHICLKHTTCFQDVVVDV
DCAENTKEDQLAEICYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYFNGHVEAVAYTVVSTVDHFVNAIEE
CGFPPTAGQPPATTKPKEITPVNPGTSPLLR
Des9_WT1
>SEQ ID NO: 18
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDORQYGDVFKGDLNPKP
QGQRLIEVSVEENHPFTLRAPIQRIYGVRYTECWSFLPSLTCTGDAAPAIQHICLKHTTCFQDVVVDV
DCAENTKEDQLACISYRFOGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYONCEHADNY
TAYCLGISHMEPSFGLILCDGGTTLCFVDTPESLSGLYVFVVYFNGHVEAVAYTVVSTVDHFVNAIEE
CGFPPTAGQPPATTKPKEITPVNPGTSPLLR
Des10_WT1
>SEQ ID NO: 19
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIECSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLCTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYONCEHADNY
TAYCLGISHMEPSFGLILCDGGTTLCFVDTPESLSGLYVFVVYFNGHVEAVAYTVVSTVDHFVNAIEE
CGFPPTAGQPPATTKPKEITPVNPGTSPLLR
Des11_WT1
>SEQ ID NO: 20
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGCRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSCGTSTYATFLVTWKGDEKTRNPTPAVTPQPRGAEFHMWNYHSHVFSVGDTESLAMHLQYKIHEA
PFDLLLEWLYVPIDPTCQPMRLYSCCLYHPNAPQCLSHMNSGCTFTSPHLAQRVASTVYONCEHADNY
TAYCLGISHMEPSFGLILCDGGTTLCFVDTPESLSGLYVFVVYFNGHVEAVAYTVVSTVDHFVNAIEE
CGFPPTAGQPPATTKPKEITPVNPGTSPLLR
Des12_WT1
>SEQ ID NO: 21
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDORQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISCRFQGKKEADQPCIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYENGHVEAVAYTVVSTVDHFVNAIEE
CGFPPTAGQPPATTKPKEITPVNPGTSPLLR
Des 13_WT1
> SEQ ID NO: 22
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPCORIYGVRYTETWSFLPSLTCTGDAAPAIQHICLKHTTCFQDVVVDV
DCAENTKEDQLCEISYRFQGKKEADQPWIVVNTSTLFDELELDPPECEPCVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYONCEHADNY
TAYCLGISHMEPSFGLILCDGGTTLCFVDTPESLSGLYVFVVYFNGHVEAVAYTVVSTVDHFVNAIEE
CGFPPTAGQPPATTKPKEITPVNPGTSPLLR
Des14_WT1
> SEQ ID NO: 23
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGVRYCETWSFLPSLTCTGDAAPAIQHICLKHTTCFQDVVVDV
DCAENTKEDQLCEICYRFQGKKEADQPWIVVNTSTLFDELELDPPECEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYONCEHADNY
TAYCLGISHMEPSFGLILCDGGTTLCFVDTPESLSGLYVFVVYFNGHVEAVAYTVVSTVDHFVNAIEE
CGFPPTAGQPPATTKPKEITPVNPGTSPLLR
Des 15_WT1
> SEQ ID NO: 24
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGVRYTECWSFLPSLTCTGDAAPAIQHICLKHTTCFQDVVVDV
DCAENTKEDQLCCISYRFOGKKEADQPWIVVNTSTLFDELELDPPECEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYENGHVEAVAYTVVSTVDHFVNAIEE
CGFPPTAGOPPATTKPKEITPVNPGTSPLLR
Des16_WT1
> SEQ ID NO: 25
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPCQRIYGVRYCETWSFLPSLTCTGDAAPAIQHICLKHTTCFQDVVVDV
DCAENTKEDQLAEICYRFQGKKEADQPWIVVNTSTLFDELELDPPEIEPCVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYONCEHADNY
TAYCLGISHMEPSFGLILCDGGTTLCFVDTPESLSGLYVFVVYENGHVEAVAYTVVSTVDHFVNAIEE
CGFPPTAGQPPATTKPKEITPVNPGTSPLLR
Des17_WT1
> SEQ ID NO: 26
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPCORIYGVRYTECWSFLPSLTCTGDAAPAIQHICLKHTTCFQDVVVDV
DCAENTKEDQLACISYRFQGKKEADQPWIVVNTSTLFDELELDPPEIEPCVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYONCEHADNY
TAYCLGISHMEPSFGLILCDGGTTLCFVDTPESLSGLYVFVVYFNGHVEAVAYTVVSTVDHFVNAIEE
CGFPPTAGQPPATTKPKEITPVNPGTSPLLR
Des 18_WT1
> SEQ ID NO: 27
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGVRYCECWSFLPSLTCTGDAAPAIQHICLKHTTCFQDVVVDV
DCAENTKEDQLACICYRFOGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAORVASTVYONCEHADNY
TAYCLGISHMEPSFGLILCDGGTTLCFVDTPESLSGLYVFVVYENGHVEAVAYTVVSTVDHFVNAIEE
CGFPPTAGQPPATTKPKEITPVNPGTSPLLR
Des1_WT2
> SEQ ID NO: 28
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSTCLYHPNAPQCLSHMNSGCTFTSPHLAQRVASTVYONCEHADNY
TAYCLGISHMEPSFGLILCDGGTTLCFVDTPESLSGLYVFVVYFNGHVEAVAYTVVSYVDHFVNAIEE
RGFPPTAGQPPATTKPKEITPVNPGTSPLIRYAAWTGGLA
Des 2_WT2
> SEQ ID NO: 29
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYONCEHADNY
TAYCLGISHMEPSFGLILHDGGTTLKFVDTPESLSGLYVFVVYENGHVEAVAYTVVSYVDHFVNAIEE
CGFPPTAGQPPATTKPKEITPVNPGTSPLIRYAAWTGGLA
Des 3_WT2
> SEQ ID NO: 30
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYONCEHADNY
TAYCLGISHMEPSFGLILCDGGTTLCFVDTPESLSGLYVFVVYENGHVEAVAYTVVSYVDHFVNAIEE
CGFPPTAGQPPATTKPKEITPVNPGTSPLIRYAAWTGGLA
Des 4_WT2
> SEQ ID NO: 31
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDOLCEISYRFQGKKEADQPWIVVNTSTLFDELELDPPECEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYONCEHADNY
TAYCLGISHMEPSFGLILCDGGTTLCFVDTPESLSGLYVFVVYFNGHVEAVAYTVVSYVDHFVNAIEE
CGFPPTAGQPPATTKPKEITPVNPGTSPLIRYAAWTGGLA
Des5_WT2
> SEQ ID NO: 32
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPCQRIYGVRYTETWSFLPSLTCTGDAAPAIQHICLKHTTCFQDVVVDV
DCAENTKEDQLAEISYRFQGKKEADQPWIVVNTSTLFDELELDPPEIEPCVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYONCEHADNY
TAYCLGISHMEPSFGLILCDGGTTLCFVDTPESLSGLYVFVVYFNGHVEAVAYTVVSYVDHFVNAIEE
CGFPPTAGQPPATTKPKEITPVNPGTSPLIRYAAWTGGLA
Des 6_WT2
> SEQ ID NO: 33
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRCYGCRYTETWSFLPSLTCTGDAAPAIQHICLKHTTCFQDVVVDV
DCAENTKEDQLAEISYRFQGKKEADOPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPOCLSHMNSGCTFTSPHLAQRVASTVYONCEHADNY
TAYCLGISHMEPSFGLILCDGGTTLCFVDTPESLSGLYVFVVYENGHVEAVAYTVVSYVDHFVNAIEE
CGFPPTAGQPPATTKPKEITPVNPGTSPLIRYAAWTGGLA
Des 7_WT2
> SEQ ID NO: 34
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRIYGVRYTETWCFLPSLTCTGDAAPAIQHICLKHTTCFQDVVVDV
DCAENTKEDCLAEISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYENGHVEAVAYTVVSYVDHFVNAIEE
CGFPPTAGQPPATTKPKEITPVNPGTSPLIRYAAWTGGLA
Des 8_WT2
> SEQ ID NO: 35
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRIYGVRYCETWSFLPSLTCTGDAAPAIQHICLKHTTCFQDVVVDV
DCAENTKEDQLAEICYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYFNGHVEAVAYTVVSYVDHFVNAIEE
CGFPPTAGQPPATTKPKEITPVNPGTSPLIRYAAWTGGLA
Des9_WT2
> SEQ ID NO: 36
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRIYGVRYTECWSFLPSLTCTGDAAPAIQHICLKHTTCFQDVVVDV
DCAENTKEDQLACISYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYENGHVEAVAYTVVSYVDHFVNAIEE
CGFPPTAGQPPATTKPKEITPVNPGTSPLIRYAAWTGGLA
Des10_WT2
> SEQ ID NO: 37
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIECSVEENHPFTLRAPIQRIYGVRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSDGTSTYATFLCTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYFNGHVEAVAYTVVSYVDHFVNAIEE
CGFPPTAGQPPATTKPKEITPVNPGTSPLIRYAAWTGGLA
Des11_WT2
> SEQ ID NO: 38
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVDQRQYGDVFKGDLNPKP
QGQRLIEVSVEENHPFTLRAPIQRIYGCRYTETWSFLPSLTCTGDAAPAIQHICLKHTTCFQDVVVDV
DCAENTKEDQLAEISYRFQGKKEADQPWIVVNTSTLFDELELDPPEIEPGVLKVLRTEKQYLGVYIWN
MRGSCGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYENGHVEAVAYTVVSTVDHFVNAIEE
CGFPPTAGQPPATTKPKEITPVNPGTSPLTRYAAWTGGLA
Des12_WT2
> SEQ ID NO: 39
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRIYGVRYTETWSFLPSLTCTGDAAPAIQHICLKHTTCFQDVVVDV
DCAENTKEDQLAEISCRFQGKKEADQPCIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYFNGHVEAVAYTVVSYVDHFVNAIEE
CGFPPTAGQPPATTKPKEITPVNPGTSPLIRYAAWTGGLA
Des13_WT2
> SEQ ID NO: 40
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPCQRIYGVRYTETWSFLPSLTCTGDAAPAIQHICLKHTTCFQDVVVDV
DCAENTKEDQLCEISYRFQGKKEADQPWIVVNTSTLFDELELDPPECEPCVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYENGHVEAVAYTVVSYVDHFVNAIEE
CGFPPTAGQPPATTKPKEITPVNPGTSPLIRYAAWTGGLA
Des14_WT2
> SEQ ID NO: 41
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRIYGVRYCETWSFLPSLTCTGDAAPAIQHICLKHTTCFQDVVVDV
DCAENTKEDQLCEICYRFQGKKEADQPWIVVNTSTLFDELELDPPECEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYFNGHVEAVAYTVVSYVDHFVNAIEE
CGFPPTAGQPPATTKPKEITPVNPGTSPLIRYAAWTGGLA
Des15_WT2
> SEQ ID NO: 42
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRIYGVRYTECWSFLPSLTCTGDAAPAIQHICLKHTTCFQDVVVDV
DCAENTKEDQLCCISYRFQGKKEADQPWIVVNTSTLFDELELDPPECEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYENGHVEAVAYTVVSYVDHFVNAIEE
CGFPPTAGQPPATTKPKEITPVNPGTSPLIRYAAWTGGLA
Des16_WT2
> SEQ ID NO: 43
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPCQRIYGVRYCETWSFLPSLTCTGDAAPAIQHICLKHTTCFQDVVVDV
DCAENTKEDQLAEICYRFQGKKEADQPWIVVNTSTLFDELELDPPEIEPCVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYENGHVEAVAYTVVSYVDHFVNAIEE
CGFPPTAGQPPATTKPKEITPVNPGTSPLIRYAAWTGGLA
Des17_WT2
> SEQ ID NO: 44
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPCQRIYGVRYTECWSFLPSLTCTGDAAPAIQHICLKHTTCFQDVVVDV
DCAENTKEDQLACISYRFQGKKEADQPWIVVNTSTLFDELELDPPEIEPCVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYENGHVEAVAYTVVSYVDHFVNAIEE
CGFPPTAGQPPATTKPKEITPVNPGTSPLIRYAAWTGGLA
Des18_WT2
> SEQ ID NO: 45
SVLRYDDFHIDEDKLDTNSVYEPYYHSDHAESSWVNRGESSRKAYDHNSPYIWPRNDYDGFLENAHEH
HGVYNQGRGIDSGERLMQPTQMSAQEDLGDDTGIHVIPTLNGDDRHKIVNVNQRQYGDVFKGDLNPKP
QGQRLIEVSVEENHPFTLRAPIQRIYGVRYCECWSFLPSLTCTGDAAPAIQHICLKHTTCFQDVVVDV
DCAENTKEDQLACICYRFQGKKEADQPWIVVNTSTLFDELELDPPEIEPGVLKVLRTEKQYLGVYIWN
MRGSDGTSTYATFLVTWKGDEKTRNPTPAVTPQPRGAEFHMWNYHSHVFSVGDTFSLAMHLQYKIHEA
PFDLLLEWLYVPIDPTCQPMRLYSCCLYHPNAPQCLSHMNSGCTFTSPHLAQRVASTVYQNCEHADNY
TAYCLGISHMEPSFGLILCDGGTTLCFVDTPESLSGLYVFVVYENGHVEAVAYTVVSYVDHFVNAIEE
CGFPPTAGQPPATTKPKEITPVNPGTSPLIRYAAWTGGLA
VZV gE ectodomain fragment comprising FcBD
> SEQ ID NO: 46
AVTPQPRGAEFHMWNYHSHVFSVGDTFSLAMHLQYKIHEAPFDLLLEWLYVPIDPTCQPMRLYSTCLY
HPNAPQCLSHMNSGCTFTSPHLAQRVASTVYQNCEHADNYTAYCLGISHMEPSFGLILHDGGTTLKFV
DTPESLSGLYVFVVYFNGHVEAVAYTVVSTVDHFVNAIEERGFPPTAGQPPATTKPKEITPVNPGTSP
LIRYAAWTGGLA

All patents and publications referred to herein are expressly incorporated by reference in their entireties.

1. A modified Varicella Zoster Virus glycoprotein E (VZV gE) protein comprising at least one cysteine pair capable of forming a non-native disulfide bridge, the at least one cysteine pair selected from the group consisting of:
a) Cys-365 and Cys-477,
b) Cys-427 and Cys-434,
c) Cys-216 and Cys-251,
d) CVs-158 and Cys-254,
e) CVs-161 and Cys-164,
f) CVs-171 and Cys-214,
g) Cys-167 and Cys-219,
h) Cys-169 and Cys-217,
i) Cys-144 and Cys-287,
j) Cys-164 and Cys-277, and
k) Cys-220 and Cys-232;
wherein the cysteines are numbered with respect to SEQ ID NO: 1.

2.-8. (canceled)

9. The modified VZV gE protein of claim 1 comprising at least two, at least three, at least four, at least five, at least six, at least seven, at least eight, at least nine, at least 10, or all 11 cysteine pairs selected from the group consisting of (a) to (k).

10. The modified VZV gE protein of claim 1 comprising at least one cysteine pair selected from (a) and (b), and at least one cysteine pair selected from (c) to (k).

11. The modified VZV gE protein of claim 1 comprising the cysteine pairs of (a) and (b), and at least one cysteine pair of (c) to (k).

12.-29. (canceled)

30. The modified VZV gE protein of claim 1, further comprising in addition to the cysteine pair, at least one cavity-filling mutation selected from the group consisting of S443Y, S443M, S443F, S443I, L442Y, L442F, L442I, F342H, F342M, F342T, F342A, G429W, G429M, T409M, T409V, T409I, T409L, T409F, A395L, A395V, A395M, A395F, 1337M, 1337F, and I337L; wherein the stabilizing point mutations are numbered with respect to SEQ ID NO: 1.

31. (canceled)

32. The modified VZV gE protein of claim 1, further comprising a least one combination of point mutations selected from the group consisting of:
a) T356R, L390W, and Y408W,
b) T356S, M379E, S381A, G382K, L390W, Q392L, Q400P, Y408W, T409W, K434V, D437N, and F479L,
c) M331V, E339D, A340E, T356R, Q358G, M379E, N380D, S381A, G382R, L390W, Q392L, Q400P, E403T, Y408W, T409W, K434V, D437N, T462I, V467L, and F479L, and
d) M331V, L390W, T462I, and F479L;
wherein the stabilizing point mutations are numbered with respect to SEQ ID NO: 1.

33. The modified VZV gE protein of claim 1 having an amino acid sequence at least 95% identical to a non-modified VZV gE protein.

34. The modified VZV gE protein of claim 33 wherein the non-modified VZV gE protein comprises an amino acid sequence selected from the group consisting of SEQ ID NOs: 1-7.

35. The modified VZV gE protein of claim 33 comprising an amino acid sequence at least 95% identical to an amino acid sequence selected from the group consisting of SEQ ID NOs: 10-27.

36. The modified VZV gE protein of claim 33 comprising an amino acid sequence at least 95% identical to an amino acid sequence selected from the group consisting of SEQ ID NOs: 28-45.

37. The modified VZV gE protein of claim 1, further comprising a signal sequence.

38. The modified VZV gE protein of claim 37, wherein the signal sequence comprises an amino acid sequence at least 95% identical to SEQ ID NO: 8.

39.-49. (canceled)

50. The modified VZV gE protein of claim 1, the at least one cysteine pair selected from the group consisting of Cys-142 and Cys-285; Cys-144 and Cys-287; Cys-146 and Cys-152; Cys-146 and Cys-261; Cys-147 and Cys-150; Cys-154 and Cys-256; Cys-156 and Cys-254; Cys-156 and Cys-158; Cys-156 and cys-271; Cys-158 and Cys-254; Cys-161 and Cys-164; Cys-164 and Cys-277; Cys-164 and Cys-275; Cys-165 and Cys-276; Cys-166 and Cys-275; Cys-167 and Cys-219; Cys-167 and Cys-274; Cys-168 and Cys-273; Cys-169 and Cys-217; Cys-169 and Cys-272; Cys-170 and Cys-215; Cys-171 and Cys-214; Cys-171 and Cys-270; Cys-173 and Cys-268; Cys-216 and Cys-251; Cys-217 and Cys-236; Cys-217 and Cys-235; Cys-220 and Cys-232; Cys-238 and Cys-240; Cys-239 and Cys-242; Cys-241 and Cys-248; Cys-241 and Cys-246; Cys-244 and Cys-246; Cys-250 and Cys-257; Cys-269 and Cys-285; Cys-271 and Cys-283; Cys-275 and Cys-277; Cys-275 and Cys-279; and Cys-289 and Cys-292, wherein the cysteines are numbered with respect to SEQ ID NO: 1.

51. A nucleic acid encoding the modified VZV gE protein of claim 1.

52. The nucleic acid of claim 51 which is DNA.

53. The nucleic acid of claim 51 which is RNA.

54. The nucleic acid of claim 53 wherein the RNA is non-replicating RNA or self-replicating RNA.

55. (canceled)

56. A host cell comprising the nucleic acid of claim 51.

57. An immunogenic composition comprising the modified VZV gE protein of claim 1.

58.-65. (canceled)

66. A method of enhancing an immune response to VZV in a subject comprising administering the modified VZV gE protein of claim 1 to the subject.

67.-76. (canceled)
